(19) United States
(12) Patent Application Publication
(10) Pub. No.: US 2011/0212891 A1
(43) Pub. Date: Sep. 1, 2011

(54) AZEPINONE DERIVATIVES

(75) Inventors: Osamu Okamoto, Ibaraki (JP); Yasuhiro Sasaki, Okinawa (JP); Hitomi Watanabe, Ibaraki (JP); Hideki Jona, Ibaraki (JP); Kevin D. Dykstra, West Milford, NJ (US)

(73) Assignee: MSD K.K.

(21) Appl. No.: 13/126,533

(22) PCT Filed: Oct. 26, 2009
(86) PCT No.: PCT/US09/62006

§ 371 Date: Apr. 28, 2011

(30) Foreign Application Priority Data
Nov. 14, 2008 (JP) 2008-291914

(51) Int. Cl. A61K 38/12; C07K 5/00; A61P 3/10

(52) U.S. Cl. 514/6.9; 530/317

(57) ABSTRACT

The present invention relates to a compound represented by formula (I): wherein R¹ represents a hydrogen atom or the like; R² and R³ represent a hydrogen atom or the like; R⁴ is a group represented by (II) R⁵ represents a phenyl group which may be substituted with a halogen or the like; m is an integer of from 1 to 3; and p is an integer of from 0 to 4; or a pharmaceutically acceptable salt thereof.

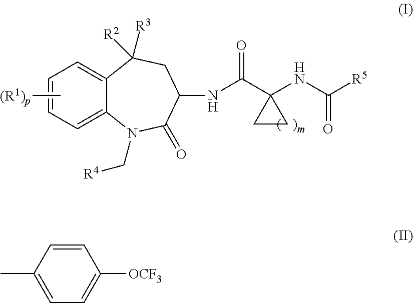

### FIELD OF THE INVENTION

The present invention relates to azepinone derivatives useful in the pharmaceutical field. These compounds have inhibitory activity of diacylglycerol O-acyltransferase type 1 (hereinafter also referred to as “DGAT1”) and useful as agents for treating and/or preventing hyperlipidemia, diabetes mellitus and obesity.

### BACKGROUND OF THE INVENTION

Obesity is a condition, in which a history of lack of exercise, intake of excessive energy, ageing, etc. leads to energy imbalance, the surplus energy is accumulated generally as neutral fat (triacylglycerol, TG) in adipose tissue, and body weight and fat mass are thus increased. In recent years, the concept of metabolic syndrome associated with obesity involving the accumulation of the visceral fat as an upstream risk factor including a plurality of risk factors of diabetes mellitus, lipidosis, hypertension, etc. has been established, and the diagnostic criteria and therapeutic guidelines for the metabolic syndrome were formulated (Journal of Japan Society for the Study of Obesity, Vol. 12, Extra Edition, 2006). Since the metabolic syndrome results in an increase of arteriosclerosis, cardiovascular disorder and cerebrovascular disorder, treatment of obesity has been recognized to be important for preventing these diseases.

Although the need of treating obesity is recognized to be important, there are extremely limited drug therapies for obesity that are currently available, and the advent of novel anti-obesity drugs having more definite action and few side-effects is thus desired.

In the living body, there are two TG synthesis pathways: a) a glycerol phosphate pathway, which is present in most organs and causes de novo TG synthesis, and b) a monoacylglycerol pathway, which is involved principally in absorption of aliphatic acid from the small intestine. Diacylglycerol acyltransferases (DGATs, EC 2.3.1.20), which are membrane-bound enzymes present in the endoplasmic reticulum, catalyze the final step of the TG synthesis common to the two TG synthesis pathways, that is, the reaction of transferring an acyl group of acyl-coenzyme A to the 3-position of 1,2-diacylglycerol to generate TG (frog. Lipid Res., 43.134-176. 2004 and Ann. Med., 36, 252-261, 2004). DGATs have been found to include two subtypes of DGATs 1 and 2. There is no significant homology at the generic or amino acid level between the DGATs 1 and 2, which are encoded by different genes (Proc. Natl. Acad. Sci. USA., 95,13018-13023,1998 and JBC, 276,38870-38876,2001). DGAT1, which is present in the small intestine, adipose tissue, the liver, etc., is believed to be involved in lipid absorption; lipid accumulation in the fat cell; and VLDL secretion and lipid accumulation in the liver, in the small intestine, the fat cell and the liver, respectively (Ann. Med., 36,252-261,2004 and JBC, 280,21506-21514,2005). In consideration of these functions of DGAT1, a DGAT1 inhibitor is expected to improve metabolic syndrome through inhibition of the lipid absorption in the small intestine, the lipid accumulation in the adipose tissue and the liver, and the lipid secretion from the liver.

In order to carry out in vivo examination of the physiological function(s) of DGAT1 and inhibitory activity against DGAT1, DGAT1-knockout mice deficient in DGAT1 at the genetic level was produced, and analyses thereof were conducted. As a result, the DGAT1-knockout mice have been found to have smaller fat masses than those of wild-type mice and to become resistant to obesity, abnormal glucose tolerance, insulin resistance and fatty liver due to a high-fat diet load (Nature Genetics, 25,87-90,2000 and JCI, 109,1049-1055,2002). In addition, energy expense has been reported to be accelerated in the DGAT1-knockout mice; and transplantation of the adipose tissues of DGAT1-knockout mice into wild-type mice has been reported to make the wild-type mice resistant to obesity and abnormal glucose tolerance, induced by a high-fat diet load (JCI, 111,1715-1722,2003 and Diabetes, 53,1445-1451,2004). In contrast, obesity and diabetes mellitus due to a high-fat diet have been reported to worsen in mice with overexpression of DGAT1 in adipose tissue (Diabetes, 51,3189-3195,2002 and Diabetes, 54,3379-3386).

From the results, DGAT1 inhibitors are likely to be therapeutic drugs with efficacy for obesity or type 2 diabetes mellitus, lipidosis, hypertension, fatty liver, arteriosclerosis, cerebrovascular disorder, coronary artery disease, or the like, associated with obesity.

A number of compounds having DGAT1 inhibitory activity are known; however, all of them have a different structure from the compounds according to the embodiments of the present invention (for example, see WO 2004/100881, WO2006/044775 and WO2006/113919).

Azepinone derivatives are disclosed in U.S. Pat. No. 6,759,404. The compounds disclosed therein inhibit generation of an Aβ peptide and thereby prevent formation of amyloid protein deposited in the nerve. U.S. Pat. No. 6,759,404 does not disclose or suggest that the azepinone derivatives are useful in treatment and/or prevention of hyperlipidemia, diabetes mellitus and obesity.

### SUMMARY OF THE INVENTION

It is desirable to provide azepinone derivatives having DGAT1 inhibitory activity.

The present inventors conducted extensive research to develop a compound having DGAT1 inhibitory activity. They found that compounds according to the embodiments of the present invention are efficacious as compounds having the DGAT1 inhibitory activity.

Specifically, the present invention relates to an agent for treating or preventing hyperlipidemia, diabetes mellitus or obesity, which contains, as an active ingredient, a compound represented by formula (I):

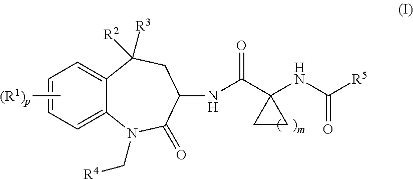

or a pharmaceutically acceptable salt thereof, wherein:
R¹ is each independently selected from the group consisting of:
   (1) halogen atom,
   (2) lower alkyl group unsubstituted or substituted with one to three halogen atoms, and
   (3) lower alkoxy group unsubstituted or substituted with one to three halogen atoms,
R² and R³ are each independently hydrogen atoms, or R² and R³ taken together form an oxo group;
R⁴ is selected from:

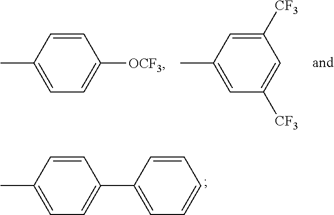

R⁵ is
   (1) a group selected from the group consisting of phenyl, pyridinyl and thiazolyl, or
   (2) a group selected from the group consisting of:

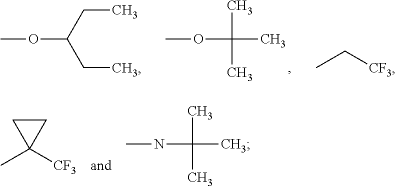

wherein phenyl, pyridinyl and thiazoly are unsubstituted or substituted with one to three halogen atoms, lower alkoxy groups or trifluoromethyl groups;
m is an integer of from 1 to three;
p is an integer of from 0 to four.

The present invention also relates to a pharmaceutical composition containing the compound represented by the formula (I) and a pharmaceutically acceptable carrier.

The present invention also relates to a DGAT1 inhibitor containing the compound represented by the formula (I) or a pharmaceutically acceptable salt thereof as an active ingredient.1

The present invention also relates to an agent for treating and/or preventing hyperlipidemia, diabetes mellitus and obesity, which contains the compound represented by the formula (I) or a pharmaceutically acceptable salt thereof as an active ingredient.

Compounds according to formula (I) of the present invention, and pharmaceutically acceptable salts thereof, have strong DGAT1 inhibitory activity and are thus useful for treating and/or preventing hyperlipidemia, diabetes mellitus and obesity.

### DETAILED DESCRIPTION OF THE INVENTION

The meanings of terms as used herein are described below, and a compound according to an embodiment of the present invention is described in further detail.

The term “halogen atom” encompasses, for example, fluorine, chlorine, bromine and iodine atoms.

The term “lower alkyl group” refers to a linear or branched C₁₋₆ alkyl group, of which examples include methyl, ethyl, propyl, isopropyl, butyl, isobutyl, sec-butyl, tert-butyl, pentyl, isoamyl, neopentyl, isopentyl, 1,1-dimethyl propyl, 1-methyl butyl, 2-methyl butyl, 1,2-dimethyl propyl, hexyl, isohexyl, 1-methyl pentyl, 2-methyl pentyl, 3-methyl pentyl, 1,1-dimethyl butyl, 1,2-dimethyl butyl, 2,2-dimethyl butyl, 1,3-dimethyl butyl, 2,3-dimethyl butyl, 3,3-dimethyl butyl, 1-ethyl butyl, 2-ethyl butyl, 1,2,2-trimethyl propyl and 1-ethyl-2-methyl propyl groups.

The term “lower alkoxy group” refers to a group, in which the hydrogen atom of a hydroxy group is substituted with the above-mentioned lower alkyl group, and of which examples include methoxy, ethoxy, propoxy, isopropoxy, butoxy, sec-butoxy, tert-butoxy, pentyloxy, isopentyloxy, hexyloxy and isohexyloxy groups.

The term “C₃₋₇ cycloalkyl group” specifically encompasses cyclopropyl, cyclobutyl, cyclopentyl, cyclohexyl and cycloheptyl groups.

In order to further specifically disclose a compound according to an embodiment of the present invention, represented by formula (I):

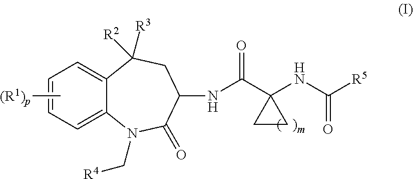

wherein each symbol has the same definition specified above, each symbol used in formula (I) is described referring to specific examples.

Each R¹ means a group independently selected from the group consisting of:
(1) hydrogen atoms;
(2) halogen atoms;
(3) lower alkyl groups which may be substituted with 1-3 halogen atoms; and
(4) lower alkoxy groups which may be substituted with 1-3 halogen atoms.

“Halogen atom” represented by R¹ encompasses identical groups as the halogen atoms defined above, of which examples specifically include fluorine, chlorine, bromine and iodine atoms.

“Lower alkyl group which may be substituted with 1-3 halogen atoms” represented by R¹ means a lower alkyl group that is unsubstituted or substituted with 1-3 halogen atoms.

The unsubstituted lower alkyl groups mean identical groups as the lower alkyl groups defined above, examples of which specifically include methyl, ethyl, n-propyl and isopropyl groups.

The lower alkyl group which may be substituted with 1-3 halogen atoms means a lower alkyl group as defined above, which is substituted with 1-3 halogen atoms which are identical or different, and specifically includes, for example, a trifluoromethyl group.

“Lower alkoxy group which may be substituted with 1-3 halogen atoms” represented by R¹ means a lower alkoxy group that is unsubstituted or substituted with 1-3 halogen atoms.

The unsubstituted lower alkoxy groups mean identical groups as the lower alkoxy groups defined above, examples of which specifically include methoxy, ethoxy, n-propoxy and isopropoxy groups.

The lower alkoxy group which may be substituted with 1-3 halogen atoms means a lower alkoxy group as defined above, which is substituted with 1-3 halogen atoms which are identical or different, and specifically includes, for example, a trifluoromethoxy group.

R² and R³ each independently represent a hydrogen atom, or R² and R³ together represent an oxo group.

R⁴ is a group selected from the group consisting of formula (II):

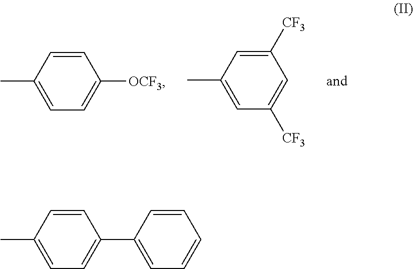

R⁴ is preferably a group represented by formula (II-1):

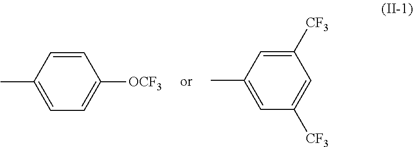

among the groups represented by the formula (II).

R⁵ is a group selected from the group consisting of: (1) phenyl, pyridinyl and thiazolyl groups which may be substituted with 1-3 halogen atoms, lower alkoxy groups or trifluoromethyl groups which are identical or different; and (2) formula (III).

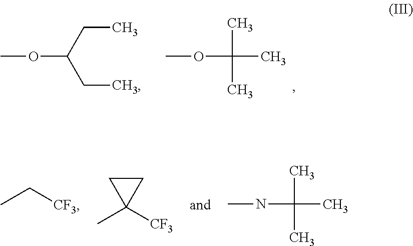

In certain embodiments R⁵ is a group selected from the group consisting of:

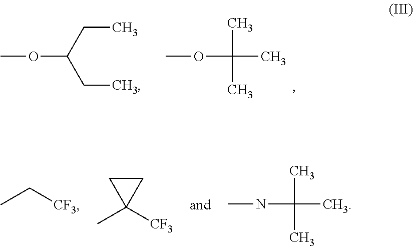

“Phenyl, pyridinyl or thiazolyl group which may be substituted with 1-3 halogen atoms, lower alkoxy groups or trifluoromethyl groups which are identical or different” represented by R⁵ refers to an unsubstituted phenyl, pyridinyl or thiazolyl group, or a phenyl, pyridinyl or thiazolyl group substituted with 1-3 halogen atoms, lower alkoxy groups or trifluoromethyl groups which are identical or different.

Halogen atoms of the substituents include identical groups as the halogen atoms defined above.

Lower alkoxy groups of the substituents include identical groups as the lower alkoxy groups defined above.

Phenyl groups substituted with 1-3 halogen atoms, lower alkoxy groups or trifluoromethyl groups which are identical or different include, for example, 4-fluorophenyl, 3-fluorophenyl, 2-fluorophenyl, 4-methoxyphenyl, 3-methoxyphenyl, 2-methoxyphenyl, 4-chlorophenyl, 3-chlorophenyl, 2-chlorophenyl, 2,4,5-trifluorophenyl and 4-fluoro-2-(trifluoromethyl)phenyl groups.

Pyridinyl groups substituted with 1-3 halogen atoms, lower alkoxy groups or trifluoromethyl groups which are identical or different include, for example, 6-fluoropyridin-2-yl, 5,6-difluoropyridin-2-yl, 6-chloro-3-fluoropyridin-2-yl, 5-fluoropyridin-2-yl, 2,6-difluoropyridin-3-yl, 6-fluoropyridin-3-yl and 2-fluoropyridin-4-yl groups.

Thiazolyl groups substituted with 1-3 halogen atoms, lower alkoxy groups or trifluoromethyl groups which are identical or different include, for example, 4-chlorothiazol-2-yl, 4-chlorothiazol-2-yl, 5-chlorothiazol-2-yl, 4-methoxythiazol-2-yl, 5-methoxythiazol-2-yl and 4,5-difluorothiazol-2-yl groups.

Specifically, examples of compounds encompassed by the present invention include, but are not limited to, tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate; tert-butyl{1-[({(3S))-1-[3,5-bis(trifluoromethyl)benzyl]-2,5-dioxo-2,3,4,5-tetrahydro-1H-1-benzazepin-3-yl}amino)carbonyl]cyclopropyl}carbamate; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluorobenzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}benzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}pyridin-2-carboxyamide; N-{(3R)-1-[3,5-bis(trifluoromethyl)benzyl]-2,5-dioxo-2,3,4,5-tetrahydro-1H-1-benzazepin-3-yl}-1-[(3,3,3-trifluoropropanoyl)amino]cyclopropane carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5,6-difluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-chloro-3-fluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-2,6-difluoronicotinamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoronicotinamide; N-{1-[({(3R)-1-[3,5-bis(trifluoromethyl)benzyl]-2,5-dioxo -2,3,4,5-tetrahydro-1H-1-benzazepin-3-yl}amino)carbonyl]cyclopropyl}-2-fluoroisonicotinamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluoro-2-(trifluoromethyl)benzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-methoxybenzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-1-(trifluoromethyl)cyclopropane carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5-fluoropyridin-2-carboxyamide; 1-ethylpropyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate; N-{(3R)-1-[3,5-bis(trifluoromethyl)benzyl]-2,5-dioxo-2,3,4,5-tetrahydro-1H-1-benzazepin-3-yl}-1-{[(tert-butyl amino)carbonyl]amino}cyclopropane carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-1,3-thiazole-2-carboxamide; tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}carbamate; N-{1-[({(3R)-1-[3,5-bis(trifluoromethyl)benzyl]-2,5-dioxo-2,3,4,5,-tetrahydro-1H-1-benzazepin-3-yl}cyclobutyl}-1,3-thiazole-2-carboxamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}-4-fluorobenzamide; tert-butyl[1-({[(3R)-1-(biphenyl-4-ylmethyl)-2,5-dioxo-2,3,4,5-tetrahydro-1H-1-benzazepin-3-yl]amino}carbonyl)cyclopropyl]carbamate; tert-butyl[1-({[(3S))-1-(biphenyl-4-ylmethyl)-2,5-dioxo-2,3,4,5-tetrahydro-1H-1-benzazepin-3-yl]amino}carbonyl)cyclopropyl]carbamate; tert-butyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate; tert-butyl{1-[({(3S))-2,5-dioxo-1-[4-(trifluoromethoxy)benzyl]-2,3,4,5-tetrahydro-1H-1-benzazepin-3-yl}amino)carbonyl]cyclopropyl}carbamate; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide; N-{1-[({(3S))-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2,4,5-trifluorobenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-4-methoxybenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}pyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-4-fluorobenzamide; 4-chloro-N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}benzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-1-(trifluoromethyl)cyclopropane carboxyamide; N-{(3R)-2,5-dioxo-1-[4-(trifluoromethoxy)benzyl]-2,3,4,5-tetrahydro-1H-1-benzazepin-3-yl}-1-[3,3,3-(trifluoropropanoyl)amino]cyclopropane carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-1,3-thiazol-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-6-fluoropyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-5,6-difluoropyridin-2-carboxyamide; 6-chloro-N-{1-[({(3R)-2,5-dioxo-1-{4-(trifluoromethoxy)benzyl]-2,3,4,5-tetrahydro-1H-1-benzazepin-3-yl}amino)carbonyl]cyclopropyl}-3-fluoropyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl benzamide; 1-ethylpropyl{1-[({(3R)-2,5-dioxo-1-[4-(trifluoromethoxy)-benzyl]-2,3,4,5-tetrahydro-1H-1-benzazepin-3-yl}amino)carbonyl]cyclopropyl}carbamate; tert-butyl{1-[({1-[3,5-bis(trifluoromethyl)benzyl)-7-fluoro-2-oxo-2,3,4,5-tetrahydro-1H-1-benzazepin-3-yl}amino)carbonyl]cyclopropyl}carbamate; N-{1-[({1-[3,5-bis(trifluoromethyl)benzyl)-7-fluoro-2-oxo-2,3,4,5-tetrahydro-1H-1-benzazepin-3-yl}amino)carbonyl]cyclopropyl}benzamide; N-[1-({[1-[3,5-bis(trifluoromethyl)benzyl)-2-oxo-7-(trifluoromethoxy)-2,3,4,5-tetrahydro-1H-1-benzazepin-3-yl]amino}carbonyl)cyclopropyl]-1-(trifluoromethyl)cyclopropane carboxyamide; N-[1-({[1-[3,5-bis(trifluoromethyl)benzyl)-2-oxo-7-(trifluoromethoxy)-2,3,4,5-tetrahydro-1H-1-benzazepin-3-yl]amino}carbonyl)cyclopropyl]benzamide; tert-butyl{1-[({1-[3,5-bis(trifluoromethyl)benzyl]-2-oxo-2,3,4,5-tetrahydro-1H-1-benzazepin-3-yl}amino)carbonyl]cyclopropyl}carbamate; N-{1-[({1-[3,5-bis(trifluoromethyl)benzyl)-2-oxo-2,3,4,5-tetrahydro-1H-1-benzazepin-3-yl}amino)carbonyl]cyclopropyl}benzamide (enantiomer A); N-{1-[({1-[3,5-bis(trifluoromethyl)benzyl]-2-oxo-2,3,4,5-tetrahydro-1H-1-benzazepin-3-yl}amino)carbonyl]cyclopropyl}-4-chlorobenzamide; tert-butyl{1-[({2-oxo-1-[4-(trifluoromethoxy)benzyl}-2,3,4,5-tetrahydro-1H-1-benzazepin-3-yl}amino)carbonyl]cyclobutyl}carbamate; tert-butyl{1-[({2-oxo-1-[4-(trifluoromethoxy)benzyl]-2,3,4,5-tetrahydro-1H-1-benzazepin-3-yl}amino)carbonyl]cyclopentyl}carbamate; N-{1-[({7-fluoro-2-oxo-1-[4-(trifluoromethoxy)benzyl]-2,3,4,5-tetrahydro-1H-1-benzazepin-3-yl}amino)carbonyl]cyclopropyl}benzamide; and N-{1-[({1-[3,5-bis(trifluoromethyl)benzyl)-6-chloro-2-oxo-2,3,4,5-tetrahydro-1H-1-benzazepin-3-yl}amino)carbonyl]cyclopropyl}benzamide.

Any preferred embodiments of R¹, R², R³, R⁴, R⁵, p and m as described above may be combined.

A process of producing a compound according to an embodiment of the present invention will now be described.

Among compounds according to an embodiment of the present invention, a compound represented by formula (I-1):

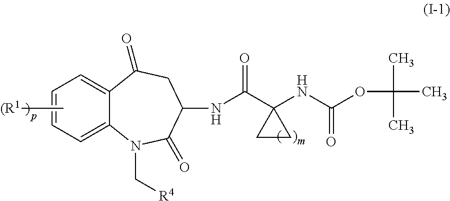

wherein each symbol has the same definition specified above, can be produced, e.g., by the following method:

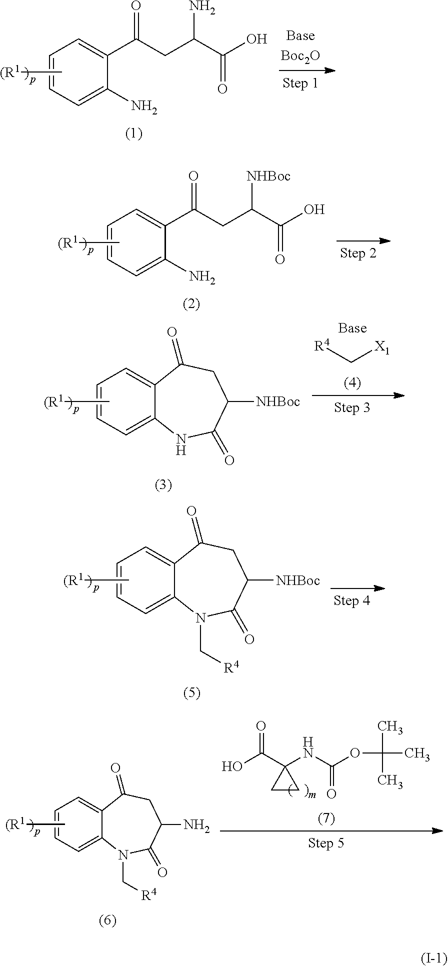

wherein X₁ is a leaving group; and the other symbols have the same definitions specified above.

### Step 1

This step is a process of producing a compound (2) by reacting a compound (1) with Boc₂O in the presence of a base.

Examples of bases as used include sodium hydrogen carbonate, potassium carbonate, triethylamine and diisopropylamine.

An amount of the base is typically 1-8 equivalents, preferably 1-4 equivalents, per equivalent of the compound (1).

The amount of Boc₂O is typically 1-3 equivalents, preferably 1-2 equivalents, per equivalent of the compound (1).

Any solvent may be used in this step unless inhibiting this reaction, examples of which include water, methanol, ethanol, tetrahydrofuran and acetonitrile, among which water and acetonitrile or mixed solvents thereof are preferred.

The reaction temperature is typically 0-80° C., preferably 10-50° C.

The reaction time is typically 1-8 hours, preferably 1-3 hours.

The compound (2) obtained in such a manner may be isolated and purified by well-known separation and purification measures such as concentration, vacuum concentration, reprecipitation, solvent extraction, crystallization and chromatography, or the isolation and purification may be omitted to subject the compound (2) to the subsequent step.

### Step 2

This step is a process of producing a compound (3) by cyclizing the compound (2) in a molecule.

For the reaction in this step, typical amide formation reaction may be performed by methods as described in documents (e.g., Nobuo Izumiya, et al.: Peptide Gosei no Kiso to Jikken (Fundamentals and Experiments of Peptide Synthesis), Maruzen (1983); Comprehensive Organic Synthesis, Vol. 6, Pergamon Press (1991), etc.), other methods known in the art or combinations thereof, that is, by using a condensation agent that is well known to those skilled in the art, or by an ester activation method that can be used by those skilled in the art, a mixed anhydride method, an acid chloride method or a carbodiimide method. Examples of such amide formation reagents include thionyl chloride, oxalyl chloride, N,N-dicyclohexylcarbodiimide, 1-methyl-2-bromopyridinium iodide, N,N′-carbonyldiimidazole, diphenylphosphoryl chloride, diphenylphosphoryl azide, N,N-disuccinimidyl carbonate, N,N-disuccinimidyl oxalate, 1-ethyl-3-(3-dimethylaminopropyl)carbodiimide hydrochloride, ethyl chloroformate, isobutyl chloroformate and benzotriazol-1-yl-oxy-tris(dimethylamino)phosphonium hexafluorophosphate; especially preferably, e.g., thionyl chloride, 1-ethyl-3-(3-dimethylaminopropyl)carbodiimide hydrochloride, N,N-dicyclohexylcarbodiimide and benzotriazol-1-yl-oxy-tris(dimethylamino)phosphonium hexafluorophosphate. For the amide formation reaction, a base and a condensation adjuvant may be also used together with the amide formation reagent.

Bases as used include ternary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-en (DBU) and 1,5-azabicyclo[4.3.0]nona-5-en (DBN); aromatic amines such as pyridine, 4-dimethylaminopyridine, picoline, lutidine, quinoline and isoquinoline; especially preferably, e.g., ternary aliphatic amines; particularly preferably, e.g., triethylamine, N,N-diisopropylethylamine, etc.

An amount of a base as used is typically 1-10 equivalents, preferably 1-5 equivalents, per equivalent of the compound (2) or a reactive derivative thereof.

Condensation adjuvants as used include, for example, N-hydroxybenzotriazole hydrate, N-hydroxy succinimide, 2,3-N-hydroxy-5-norbornen-dicarboximide and 3-hydroxy-3,4-dihydro-4-oxo-1,2,3-benzotriazole; especially preferably, e.g., N-hydroxybenzotriazole, etc.

An amount of the condensation adjuvant is typically 1-10 equivalents, preferably 1-2 equivalents, per equivalent of the compound (2) or a reactive derivative thereof.

Reaction solvents as used in this step include, but, unless interfering with the reaction, are not limited to, e.g., inert solvents; specifically, e,g., water, DMF, methylene chloride, chloroform, 2-dichloroethane, dimethylformamide, ethyl acetate, methyl acetate, acetonitrile, benzene, xylene, toluene, 1,4-dioxane, tetrahydrofuran and dimethoxyethane or mixed solvents thereof; preferably, e.g., water, methylene chloride, chloroform, 2-dichloroethane, acetonitrile and N,N-dimethylformamide or mixed solvents thereof, from the viewpoint of ensuring reaction temperature.

The reaction time is typically 1-24 hours, preferably 1-12 hours.

The reaction temperature is typically from 0° C. to the boiling point of a solvent, preferably from room temperature to 80° C.

One or a combination of two or more of bases, amide formation reagents and condensation adjuvants as used in this step may be used.

The compound (3) obtained in such a manner may be isolated and purified by well-known separation and purification measures such as concentration, vacuum concentration, crystallization, solvent extraction, reprecipitation and chromatography, or the isolation and purification may be omitted to subject the compound (3) to the subsequent step.

### Step 3

This step is a process of producing a compound (5) by reacting the compound (3) with the compound (4) in the presence of a base.

Bases used in this step can include, for example, sodium carbonate, potassium carbonate, cesium carbonate, DBU, potassium tert-butoxide and sodium tert-pentoxide, among which, e.g., potassium carbonate and sodium tert-pentoxide are preferred.

An amount of the base is typically 1-3 equivalents, preferably 1-1.5 equivalents, per equivalent of the compound (3).

X₁ represents a leaving group; and any leaving group may be used if generating the compound (5) by reaction between the compound (3) and the compound (4), of which examples specifically include halogen atoms, etc.

The amount of the compound (4) is typically 1-2 equivalents, preferably 1-1.3 equivalents, per equivalent of the compound (3).

The reaction time is typically 1-24 hours, preferably 1-12 hours.

The reaction temperature is typically from 0° C. to the boiling point of a solvent, preferably from 0° C. to room temperature.

The compound (5) obtained in such a manner may be isolated and purified by well-known separation and purification measures such as concentration, vacuum concentration, reprecipitation, solvent extraction, crystallization and chromatography, or the isolation and purification may be omitted to subject the compound (5) to the subsequent step.

### Step 4

This step is a process of producing a compound (6) by removing the Boc group of the compound (5).

The reaction in this step can be carried out by methods as described in documents (e.g., T. W. Green: Protective Groups in Organic Synthesis, Second Edition, John Wiley & Sons (1991), etc.), other methods known in the art or combinations thereof. For example, the compound (6) can be produced by adding TFA to the compound (5) dissolved in chloroform or the like.

The compound (6) obtained in such a manner may be isolated and purified by well-known separation and purification measures such as concentration, vacuum concentration, reprecipitation, solvent extraction, crystallization and chromatography, or the isolation and purification may be omitted to subject the compound (6) to the subsequent step.

### Step 5

This step is a process of producing a compound (I-1) according to an embodiment of the present invention by reacting the compound (6) with the compound (7).

The reaction in this step is an amide formation reaction, and the compound (I-1) can be produced by the methods as in the Step 2, other methods known in the art or combinations thereof, using the compounds (6) and (7).

The compound (I-1) obtained in such a manner may be isolated and purified by well-known separation and purification measures such as concentration, vacuum concentration, reprecipitation, solvent extraction, crystallization and chromatography.

Furthermore, in the compounds according to an embodiment of the present invention, a compound represented by formula (I-2):

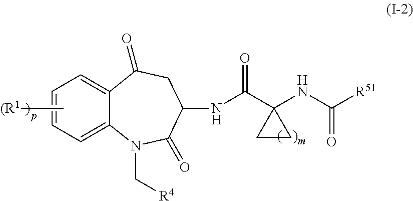

wherein each symbol has the same definition specified above, when R⁵ is R⁵¹ (R⁵¹ is (1) a phenyl, pyridinyl or thiazolyl group which may be substituted with 1-3 halogen atoms, lower alkoxy groups or trifluoromethyl groups; or a 2,2,2-trifluoroethyl, 1-(trifluoromethyl)cyclopropyl or tert-butyl amino group) in the formula (I), can be produced, e.g., by the following method.

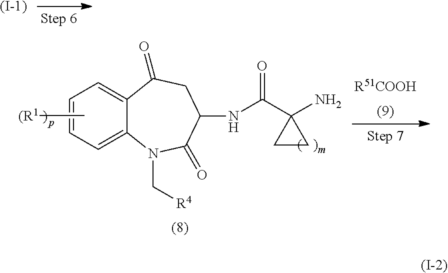

### Step 6

This step is a process of producing a compound (8) by removing the Boc group of the compound (I-1).

The reaction in this step can be carried out by methods as described in documents (e.g., T. W. Green: Protective Groups in Organic Synthesis, Second Edition, John Wiley & Sons (1991), etc.), other methods known in the art or combinations thereof. For example, the compound (8) can be produced by adding TFA to the compound (I-1) dissolved in chloroform or the like.

The compound (8) obtained in such a manner may be isolated and purified by well-known separation and purification measures such as concentration, vacuum concentration, reprecipitation, solvent extraction, crystallization and chromatography, or the isolation and purification may be omitted to subject the compound (6) to the subsequent step.

### Step 7

This step is a process of producing a compound (I-2) according to an embodiment of the present invention by reacting the compound (8) with the compound (9).

For the reaction in this step, typical amide formation reaction may be performed by methods as described in documents (e.g., Nobuo Izumiya, et al.: Peptide Gosei no Kiso to Jikken (Fundamentals and Experiments of Peptide Synthesis), Maruzen (1983); Comprehensive Organic Synthesis, Vol. 6, Pergamon Press (1991), etc.), other methods known in the art or combinations thereof, that is, by using a condensation agent that is well known to those skilled in the art, or by an ester activation method that can be used by those skilled in the art, a mixed anhydride method, an acid chloride method or a carbodiimide method. Examples of such amide formation reagents include thionyl chloride, oxalyl chloride, N,N-dicyclohexylcarbodiimide, 1-methyl-2-bromopyridinium iodide, N,N′-carbonyldiimidazole, diphenylphosphoryl chloride, diphenylphosphoryl azide, N,N-disuccinimidyl carbonate, N,N-disuccinimidyl oxalate, 1-ethyl-3-(3-dimethylaminopropyl)carbodiimide hydrochloride, ethyl chloroformate, isobutyl chloroformate and benzotriazol-1-yl-oxy-tris(dimethylamino)phosphonium hexafluorophosphate; especially preferably, e.g., thionyl chloride, 1-ethyl-3-(3-dimethylaminopropyl)carbodiimide hydrochloride, N,N-dicyclohexylcarbodiimide and benzotriazol-1-yl-oxy-tris(dimethylamino)phosphonium hexafluorophosphate. For the amide formation reaction, a base and a condensation adjuvant may be also used together with the amide formation reagent.

Bases as used include ternary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-en (DBU) and 1,5-azabicyclo[4.3.0]nona-5-en (DBN); aromatic amines such as pyridine, 4-dimethylaminopyridine, picoline, lutidine, quinoline and isoquinoline; especially preferably, e.g., ternary aliphatic amines; particularly preferably, e.g., triethylamine, N,N-diisopropylethylamine, etc.

An amount of a base as used is typically 1-10 equivalents, preferably 1-5 equivalents, per equivalent of the compound (9) or a reactive derivative thereof.

Condensation adjuvants as used include, for example, N-hydroxybenzotriazole hydrate, N-hydroxy succinimide, 2,3-N-hydroxy-5-norbornen-dicarboximide and 3-hydroxy-3,4-dihydro-4-oxo-1,2,3-benzotriazole; especially preferably, e.g., N-hydroxybenzotriazole, etc.

An amount of the condensation adjuvant is typically 1-10 equivalents, preferably 1-2 equivalents, per equivalent of the compound (9) or a reactive derivative thereof.

An amount of the compound (8) used is typically 1-10 equivalents, preferably 1-2 equivalents, per equivalent of the compound (9) or a reactive derivative thereof.

Reaction solvents as used in this step include, but, unless interfering with the reaction, are not limited to, e.g., inert solvents; specifically, e.g., DMF, methylene chloride, chloroform, 1,2-dichloroethane, dimethylformamide, ethyl acetate, methyl acetate, acetonitrile, benzene, xylene, toluene, 1,4-dioxane, tetrahydrofuran and dimethoxyethane or mixed solvents thereof; preferably, e.g., methylene chloride, chloroform, 2-dichloroethane, acetonitrile and N,N-dimethylformamide from the viewpoint of ensuring reaction temperature.

The reaction time is typically 1-24 hours, preferably 1-12 hours.

The reaction temperature is typically from 0° C. to the boiling point of a solvent, preferably from room temperature to 80° C.

One or a combination of two or more of bases, amide formation reagents and condensation adjuvants as used in this step may be used.

The compound (I-2) obtained in such a manner may be isolated and purified by well-known separation and purification measures such as concentration, vacuum concentration, crystallization, solvent extraction, reprecipitation and chromatography.

Furthermore, in the compounds according to an embodiment of the present invention, a compound represented by formula (I-3):

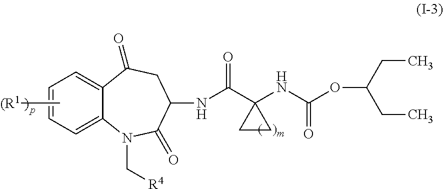

wherein each symbol has the same definition specified above, when R⁵ is 1-ethyl propoxy in the formula (I), can be produced, e.g., by the following method.

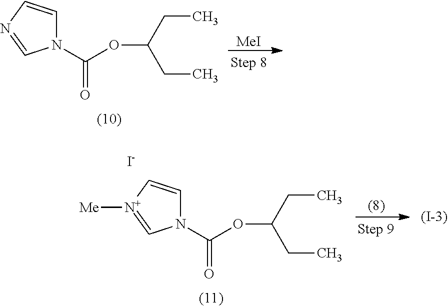

### Step 8

This step is a process of producing a compound (11) by reacting 1-ethyl propyl 1H-imidazol-1-carboxylate (10) with methyl iodide.

An amount of methyl iodide as used in this step is typically 1-50 equivalents, preferably 5-10 equivalents, per equivalent of the compound (10).

The reaction time is typically 1-48 hours, preferably 4-24 hours.

The reaction temperature is typically from room temperature to the boiling point of a solvent, preferably from the room temperature to 40° C.

Any reaction solvent may be used unless inhibiting the reaction in this step, examples of which include acetonitrile, tetrahydrofuran and chloroform.

The compound (11) obtained in such a manner may be isolated and purified by well-known separation and purification measures such as concentration, vacuum concentration, reprecipitation, solvent extraction, crystallization and chromatography, or the isolation and purification may be omitted to subject the compound (11) to the subsequent step.

### Step 9

This step is a process of producing a compound (I-3) by reacting the compound (11) with the compound (8).

An amount of the compound (11) as used in this step is typically 1-10 equivalents, preferably 1-5 equivalents, per equivalent of the compound (8).

The reaction time is typically 1-24 hours, preferably 1-12 hours.

The reaction temperature is typically from 0° C. to the boiling point of a solvent, preferably from room temperature to 50° C.

The compound (I-3) obtained in such a manner may be isolated and purified by well-known separation and purification measures such as concentration, vacuum concentration, reprecipitation, solvent extraction, crystallization and chromatography.

Furthermore, in the compounds according to an embodiment of the present invention, a compound represented by formula (I-4):

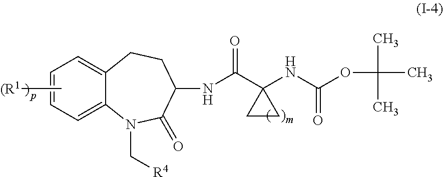

wherein each symbol has the same definition specified above, can be produced, e.g., by the following method:

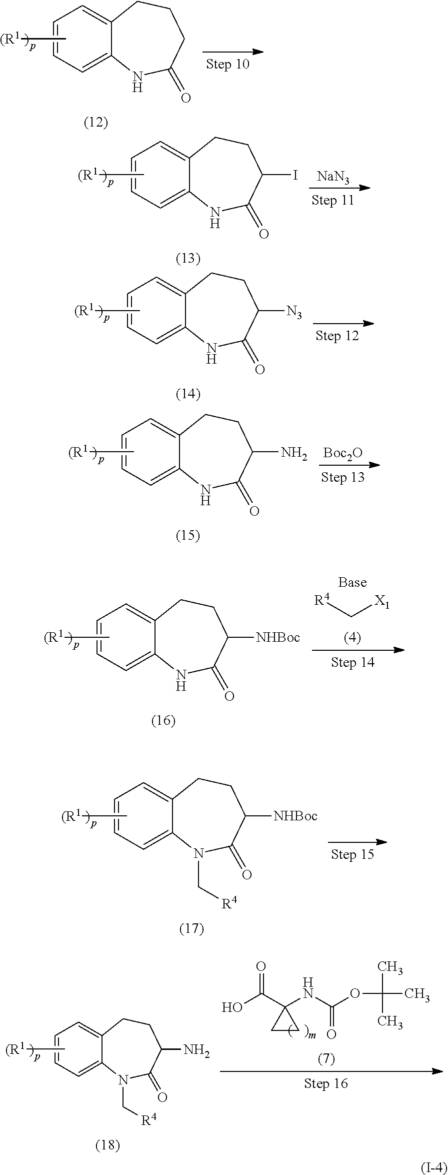

wherein each symbol has the same definition specified above.

### Step 10

This step is a process of producing a compound (13) by reacting the compound (12) with iodine in the presence of a base and iodotrimethylsilane.

Bases used in this step can include, for example, ethyl diisopropylamine, 2,4,6-collodine and tetramethylethylenediamine.

An amount of the base is typically 1-10 equivalents, preferably 1-5 equivalents, per equivalent of the compound (12).

An amount of iodotrimethylsilane as used in this step is typically 1-5 equivalents, preferably 1-3 equivalents, per equivalent of the compound (12).

An amount of iodine as used in this step is typically 1-5 equivalents, preferably 1-2 equivalents, per equivalent of the compound (12).

The reaction time is typically from 30 minutes to 3 hours, preferably from 30 minutes to 2 hours.

The reaction temperature is typically from −40° C. to 0° C., preferably from −20° C. to 0° C.

Any reaction solvent may be used unless inhibiting the reaction in this step, examples of which include dichloromethane, toluene, N,N-dimethylformamide and acetonitrile.

The compound (13) obtained in such a manner may be isolated and purified by well-known separation and purification measures such as concentration, vacuum concentration, reprecipitation, solvent extraction, crystallization and chromatography, or the isolation and purification may be omitted to subject the compound (13) to the subsequent step.

### Step 11

This step is a process of producing a compound (14) by reacting the compound (13) with sodium azide.

An amount of sodium azide is typically 1-10 equivalents, preferably 2-5 equivalents, per equivalent of the compound (13).

The reaction time is typically from 1-24 hours, preferably 1-12 hours.

The reaction temperature is typically from 0° C. to the boiling point of a solvent, preferably from room temperature to 50° C.

Any reaction solvent may be used unless inhibiting the reaction in this step, examples of which include dimethyl sulfoxide.

The compound (14) obtained in such a manner may be isolated and purified by well-known separation and purification measures such as concentration, vacuum concentration, reprecipitation, solvent extraction, crystallization and chromatography, or the isolation and purification may be omitted to subject the compound (14) to the subsequent step.

### Step 12

This step is a process of producing a compound (15) by reducing the compound (14).

Reductive reactions in this step, for which methods known to those skilled in the art may be used, include, for example, a reductive reaction using palladium carbon under hydrogen atmosphere.

An amount of palladium carbon as used is typically 0.01-2 equivalents, preferably 0.1-0.5 equivalent, per equivalent of the compound (14).

The reaction time is typically from 1-24 hours, preferably 1-12 hours.

The reaction temperature is typically from 0° C. to 50° C., preferably from room temperature to 50° C.

Any reaction solvent may be used unless inhibiting the reaction in this step, examples of which include methanol, ethanol and chloroform or mixed solvents thereof.

The compound (15) obtained in such a manner may be isolated and purified by well-known separation and purification measures such as concentration, vacuum concentration, reprecipitation, solvent extraction, crystallization and chromatography, or the isolation and purification may be omitted to subject the compound (15) to the subsequent step.

### Step 13

This step is a process of producing a compound (16) by introducing a Boc group into the amino group of the compound (15).

The reaction in this step may be carried out by the methods as in the Step 1, other methods known in the art or combinations thereof.

The compound (16) obtained in such a manner may be isolated and purified by well-known separation and purification measures such as concentration, vacuum concentration, reprecipitation, solvent extraction, crystallization and chromatography, or the isolation and purification may be omitted to subject the compound (16) to the subsequent step.

### Step 14

This step is a process of producing a compound (17) by reacting the compound (16) with the compound (4) in the presence of a base.

The reaction in this step may be carried out by the methods as in the Step 3, other methods known in the art or combinations thereof.

The compound (17) obtained in such a manner may be isolated and purified by well-known separation and purification measures such as concentration, vacuum concentration, reprecipitation, solvent extraction, crystallization and chromatography, or the isolation and purification may be omitted to subject the compound (17) to the subsequent step.

### Step 15

This step is a process of producing a compound (18) by removing a Boc group of the compound (17).

The reaction in this step may be carried out by the methods as in the Step 4, other methods known in the art or combinations thereof.

The compound (18) obtained in such a manner may be isolated and purified by well-known separation and purification measures such as concentration, vacuum concentration, reprecipitation, solvent extraction, crystallization and chromatography, or the isolation and purification may be omitted to subject the compound (18) to the subsequent step.

### Step 16

This step is a process of producing a compound (I-4) according to an embodiment of the present invention by reacting the compound (18) with the compound (7).

The reaction in this step may be carried out by the methods as in the Step 5, other methods known in the art or combinations thereof.

The compound (I-4) obtained in such a manner may be isolated and purified by well-known separation and purification measures such as concentration, vacuum concentration, reprecipitation, solvent extraction, crystallization and chromatography.

Furthermore, in the compounds according to an embodiment of the present invention, a compound represented by formula (I-5):

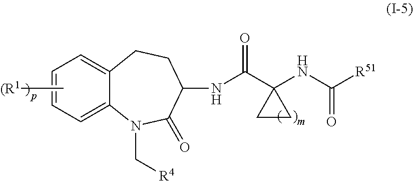

wherein each symbol has the same definition specified above, can be produced, e.g., by the following method.

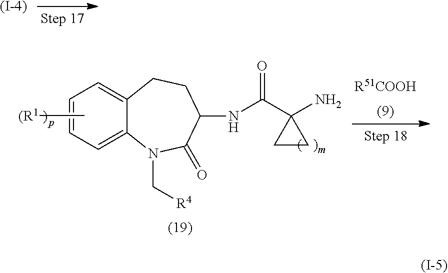

### Step 17

This step is a process of producing a compound (19) by removing a Boc group of the compound (I-4).

The reaction in this step may be carried out by the methods as in the Step 6, other methods known in the art or combinations thereof.

The compound (19) obtained in such a manner may be isolated and purified by well-known separation and purification measures such as concentration, vacuum concentration, reprecipitation, solvent extraction, crystallization and chromatography, or the isolation and purification may be omitted to subject the compound (19) to the subsequent step.

### Step 18

This step is a process of producing a compound (I-5) according to an embodiment of the present invention by reacting the compound (19) with the compound (9).

The reaction in this step may be carried out by the methods as in the Step 7, other methods known in the art or combinations thereof.

The compound (I-5) obtained in such a manner may be isolated and purified by well-known separation and purification measures such as concentration, vacuum concentration, reprecipitation, solvent extraction, crystallization and chromatography.

Furthermore, in the compounds according to an embodiment of the present invention, a compound represented by formula (I-6):

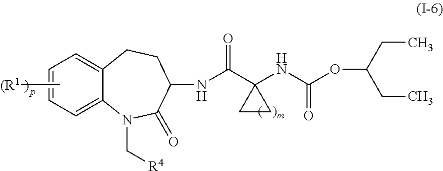

can be produced, e.g., by the following method.

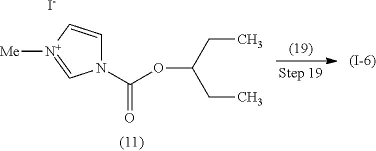

### Step 19

This step is a process of producing a compound (I-6) according to an embodiment of the present invention by reacting the compound (11) with the compound (19).

The reaction in this step may be carried out by the methods as in the Step 9, other methods known in the art or combinations thereof.

The compound (I-6) obtained in such a manner may be isolated and purified by well-known separation and purification measures such as concentration, vacuum concentration, reprecipitation, solvent extraction, crystallization and chromatography.

The azepinone derivatives in accordance with an embodiment of the present invention may be present as pharmaceutically acceptable salts, which may be produced according to methods known in the art using the compound represented by the formula (I).

Examples of such acid addition salts include hydrohalic acid salts such as hydrochloride, hydrofluorate, hydrobromide and hydroiodide; inorganic acid salts such as nitride, perchlorate, sulfate, phosphate and carbonate; lower alkyl sulfonate salts such as methanesulfonate, trifluoromethanesulfonate and ethanesulfonate; aryl sulfonates such as benzensulphonate and p-toluenesulfonate; organic salts such as fumarate, succinate, citrate, tartrate, oxalate and maleate; and acid addition salts of organic acids, e.g., amino acids, such as glutamate and aspartate.

When the compound according to an embodiment of the present invention has an acidic group, such as carboxyl, in the group, the compound can be also converted into a corresponding pharmaceutically acceptable salt by processing the compound with a base. Examples of such base addition salts include alkali metal salts such as sodium and potassium; alkaline earth metal salts such as calcium and magnesium; ammonium salts; and salts of organic bases such as guanidine, triethylamine and dicyclohexylamine.

Furthermore, the compound according to an embodiment of the present invention may be present in the form of a free compound or any hydrate or solvate of a salt thereof.

In contrast, a salt or ester can be also converted into a free compound by a usual method.

Furthermore, in the compound according to an embodiment of the present invention, a stereoisomer or a tautomer, such as an optical isomer, a diastereoisomer or a geometrical isomer, is sometimes present depending on the form of a substituent. It will be appreciated that these isomers are encompassed entirely by compounds according to an embodiment of the present invention. Furthermore, it will be appreciated that any mixture of these isomers is encompassed by compounds according to an embodiment of the present invention.

A compound represented by general formula (I) may be orally or parenterally administered and is formulated into a form suitable for such administration to provide an agent for treating and/or preventing hyperlipidemia, diabetes mellitus and obesity using the compound.

When the compound according to an embodiment of the present invention is clinically used, a pharmaceutically acceptable additive may be also added, depending on a dosage form, to produce various preparations, followed by administration of the preparations. Additives in this case, for which various additives that are usually used in the field of formulation, include, for example, gelatine, lactose, saccharose, titanium oxide, starch, microcrystalline cellulose, hydroxypropyl methylcellulose, carboxymethyl cellulose, corn starch, microcrystalline wax, white petrolatum, magnesium aluminometasilicate, anhydrous calcium phosphate, citric acid, trisodium citrate, hydroxypropylcellulose, sorbitol, sorbitan fatty acid esters, polysorbates, sucrose fatty acid esters, polyoxyethylene, hydrogenated castor oil, polyvinyl pyrrolidone, magnesium stearate, light anhydrous silicic acid, talc, vegetable oil, benzyl alcohol, gum arabic, propylene glycol, polyalkylene glycol, cyclodextrin, hydroxypropyl cyclodextrin, etc.

Examples of dosage forms as formulated mixtures with such additives include solid preparations such as tablets, capsules, granules, powders and suppositories; and liquid preparations such as syrups, elixirs and injectables, which can be prepared according to typical methods in the field of formulation. Further, the liquid preparations may be in the form of dissolution or suspension in water or another appropriate medium just before use. Particularly, the injectables may be also dissolved or suspended in a physiological saline solution or a glucose solution as needed, and a buffer or a preservative may be further added to the mixture.

Such preparations may contain the compound according to an embodiment of the present invention at a rate of L0-100%, preferably 1.0-60%, by weight of the total drug. Such preparations may also contain other therapeutically-effective compounds.

The compound according to an embodiment of the present invention may be used in combination with a drug efficacious for hyperlipidemia, diabetes mellitus, obesity or the like (hereinafter referred to as “concomitant drug”). Such drugs may be administered concurrently, separately or sequentially in treatment or prevention of the diseases. When the compound according to an embodiment of the present invention is used concurrently with one or more concomitant drugs, they may be formed into a pharmaceutical composition in a single dosage form. In a combination therapy, however, a composition containing the compound according to an embodiment of the present invention and a concomitant drug in different packages may be administered concurrently, separately or sequentially to an administration subject. They may be also administered at intervals.

A dose of a concomitant drug may be based on a dose which is clinically used and may be selected appropriately depending on an administration subject, an administration route, a disease, a combination and the like. A dosage loan of such a concomitant drug is not particularly limited, and it may be any form in which the compound according to an embodiment of the present invention and a concomitant drug are combined when they are administered. Examples of such dosage fowls include (1) administration of a single pharmaceutical preparation obtained by formulating the compound according to an embodiment of the present invention and a concomitant drug concurrently; (2) coadministration via the same administration route of two pharmaceutical preparations obtained by formulating the compound according to an embodiment of the present invention and a concomitant drug separately; (3) administration at an interval via the same administration route of two pharmaceutical preparations obtained by formulating the compound according to an embodiment of the present invention and a concomitant drug separately; (4) coadministration via different administration routes of two pharmaceutical preparations obtained by formulating the compound according to an embodiment of the present invention and a concomitant drug separately; and (5) administration at an interval via different administration routes of two pharmaceutical preparations obtained by formulating the compound according to an embodiment of the present invention and a concomitant drug separately (e.g. administration of the compound according to an embodiment of the present invention and then a concomitant drug, or administration in the reverse order). The blending ratio of the compound according to an embodiment of the present invention and a concomitant drug may be selected appropriately depending on an administration subject, an administration route, a disease, and the like.

When the compound according to an embodiment of the present invention is used in clinical fields, a dosage regimen of it depends on the sex, age, body weight and severity of condition of a patient; and the type and range of desired therapeutic effect. In case of oral administration to an adult human, the usual dosage regimen of it is 0.01-100 mg/kg per day, preferably 0.03-1 mg/kg per day in one dose or several divided doses. In case of parenteral administration, it is 0.001-10 mg/kg per day, preferably 0.001-0.1 mg/kg per day in one dose or several divided doses.

Any appropriate administration route may be used to administer an effective amount of the compound according to an embodiment of the present invention to a mammal, particularly to a human. For example, oral, rectum, local, intravenous, ocular, lung and nasal administration routes may be used. Examples of dosage forms include tablets, troches, powders, suspensions, solutions, capsules, creams, aerosols, etc., in which tablets for oral use are preferred.

For preparing compositions for oral use, any typical pharmaceutical medium may be used, examples of which include water, glycol, oils, alcohols, flavoring agents, preservatives, coloring agents. For preparing liquid compositions for oral use, examples of pharmaceutical media include suspensions, elixirs and solutions, and examples of carriers include starches, sugars, microcrystalline celluloses, diluents, granulating agents, lubricants, binders and disintegrating agents. For preparing solid compositions for oral use, examples of pharmaceutical media include powders, capsules and tablets. Particularly, the solid compositions for oral use are preferred.

Because of their ease of administration, tablets and capsules represent the most advantageous oral dosage unit form. If desired, tablets can be coated with standard aqueous or non-aqueous techniques.

In addition to the common dosage forms described above, the compounds according to formula (I) may also be administered by controlled release means and/or delivery devices that are described in U.S. Pat. Nos. 3,845,770; 3,916,899; 3,536,809; 3,598,123; 3,630,200 and 4,008,719.

Pharmaceutical compositions in accordance with an embodiment of the present invention suitable for oral administration include capsules, cachets or tablets, each containing a predetermined amount of an active ingredient, such as a powder or granules, or as an aqueous liquid, a non-aqueous liquid, an oil-in-water emulsion or a water-in-oil liquid emulsion. Such compositions may be prepared by any pharmaceutical method, including a method of combining an active ingredient with a carrier consisting of one or more necessary constituents.

In general, compositions are prepared by uniformly and sufficiently mixing active ingredients with liquid carriers or finely divided solid carriers, or both, and then shaping the product into the desired form if necessary. For example, a tablet can be prepared optionally together with one or more accessory ingredients by compression or molding. Compressed tablets can be prepared by compressing, in a suitable machine, the active ingredients in a free-flowing form such as powder or granules, optionally mixed with a binder, a lubricant, an inert excipient, a surfactant or a dispersive agent.

Molded tablets can be prepared by molding, in a suitable machine, a mixture of the powdered compound moistened with an inert liquid diluent.

Preferably, each tablet contains about 1 mg to 1 g of active ingredient, and each cachet or capsule contains about 1 mg to 500 mg of active ingredient.

Examples of pharmaceutical dosage forms for the compound of formula (I) are shown below.

TABLE 1
Suspension for Injection (I.M.)

| | mg/ml |
|---|---|
| Compound of formula | 10 |
| (I) | |
| Methyl cellulose | 5.0 |
| Tween 80 | 0.5 |
| Benzyl alcohol | 9.0 |
| Benzalkonium | 1.0 |
| chloride | |

Adjusted to 1.0 ml by addition of water for injection.

TABLE 2
Tablet

| | mg/tablet |
|---|---|
| Compound of formula | 25 |
| (I) | |
| Methyl cellulose | 415 |
| Tween 80 | 14.0 |
| Benzyl alcohol | 43.5 |
| Magnesium stearate | 2.5 |
| Total | 500 mg |

TABLE 3
Capsule

| | mg/capsule |
|---|---|
| Compound of formula | 25 |
| (I) | |
| Lactose powder | 573.5 |
| Magnesium stearate | 1.5 |
| Total | 600 mg |

TABLE 4
Aerosol

| | Per container | |
|---|---|---|
| Compound of formula (I) | 24 | mg |
| Lecithin, NF Liq. Conc | 1.2 | mg |
| Trichlorofluoromethane, NF | 4.025 | g |
| Dichlorodifluoromethane, NF | 12.15 | g |

The compound of formula (I) may be used in combination with other drugs used in treatment/prevention/delay of onset of hyperlipidemia, diabetes mellitus or obesity as well as diseases or conditions associated therewith. The other drugs may be administered in an administration route or a dose that is typically used, concurrently with or separately from the compound of formula (I).

When the compound of formula (I) is used concurrently with one or more drugs, a pharmaceutical composition containing the compound of the formula (I) and the other drugs is preferred.

Accordingly, the pharmaceutical composition according to an embodiment of the present invention contains the compound of formula (I) as well as other active ingredients that are one or more. Examples of active ingredients which are used in combination with the compound of formula (I) include, but are not limited to, the following (a) to (i):
(a) other DGAT1 inhibitors;
(b) glucokinase activators;
(C) biguanides (e.g., buformin, metformin and phenformin);
(d) PPAR agonists (e.g., troglitazone, pioglitazone and rosiglitazone);
(e) insulin;
(f) somatostatin;
(g) α-glucosidase inhibitors (e.g., voglibose, miglitol and acarbose);
(h) insulin secretagogues (e.g., acetohexamide, carbutamide, chlorpropamide, glybenclamide, gliclazide, glimepiride, glipizide, gliquidone, glisoxepide, glyburide, glyhexamide, glypinamide, phenbutamide, tolazamide, tolbutamide, tolcyclamide, nateglinide and repaglinide);
(i) DPP-IV (dipeptidyl peptidase-IV) inhibitors; and
(j) glucose uptake facilitators,
   which may be administered separately or in the same pharmaceutical composition.

A weight ratio of the compound of formula (I) to a second active ingredient varies within wide limits and further depends on the effective dose of each active ingredient. Accordingly, for example, when the compound of formula (I) is used in combination with a PPAR agonist, a weight ratio of the compound of formula (I) to the PPAR agonist is generally about 1000:1 to 1:1000, preferably about 200:1 to 1:200. Combinations of the compound of formula (I) and other active ingredients are within the above-mentioned range; and in any case, the effective dose of each active ingredient should be used.

The present invention is described below in more detail referring to Examples and Reference Examples, but is not limited thereto.

The compound according to an embodiment of the present invention or a pharmaceutically acceptable salt thereof has strong DGAT1 inhibitory activity and is thus useful for treating and/or preventing hyperlipidemia, diabetes mellitus and obesity.

It should be understood by those skilled in the art that various modifications, combinations, sub-combinations and alterations may occur depending on design requirements and other factors insofar as they are within the scope of the appended claims or the equivalents thereof.

### EXAMPLES

Wakogel (registered trademark) C-300, made by Wako Pure Chemical Industries Ltd., or KP-Sil (registered trademark) Silica prepacked column, made by Biotage, was used for the silica gel column chromatography in Examples. Kieselgel™ 60 F₂₅₄, Art. 5744, made by Merck & Co., was used for preparative thin layer chromatography. Chromatorex (registered trademark) NH (100-250 mesh or 200-350 mesh), made by Fuji Silysia Chemical Ltd., was used for basic silica gel column chromatography.

¹H-NMR was measured using Gemini (200 MHz, 300 MHz), Mercury (400 MHz) and Inova (400 MHz), made by Varian, using tetramethylsilane as a standard substance. In addition, the mass spectra were measured by electrospray ionization (ESI) or atmospheric pressure chemical ionization (APCI) using Micromass ZQ made by Waters.

The meanings of the abbreviations in Examples are shown below.
i-Bu=isobutyl
n-Bu=n-butyl
t-Bu=tert-butyl
Boc=tert-butoxycarbonyl
Me=methyl
Et=ethyl
Ph=phenyl
i-Pr=isopropyl
n-Pr=n-propyl
CDCl₃=heavy chloroform
CD₃OD=heavy methanol
DMSO-d₆=heavy dimethylsulfoxide

The meanings of the abbreviations in the nuclear magnetic resonance spectra are shown below.
s=singlet
d=doublet
dd=double doublet
dt=double triplet
ddd=double double doublet
Sept=septet
t=triplet
m=multiplet
br=broad
brs=broad singlet
q=quartet
J=coupling constant
Herz=hertz

### Example 1

### Synthesis of tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate

### Step 1

### Synthesis of tert-butyl{(3R)-1-[3,5-bis(trifluoromethyl)benzyl]-2,5-dioxo-2,3,4,5-tetrahydro-1H-1-benzazepin-3-yl}carbamate

Sodium-tert-pentoxide and 3,5-bis(trifluoromethyl)benzyl bromide were added to a solution of tert-butyl[(3R)-2,5-dioxo-2,3,4,5-tetrahydro-1H-1-benzazepin-3-yl]carbamate in N,N-dimethylformamide obtained in Reference Example 1 at −15° C., and the mixture was stirred at −15° C. for 30 minutes. Furthermore, N,N-dimethylformamide and 3,5-bis(trifluoromethyl)benzyl bromide were added, and the mixture was stirred at −15° C. for 30 minutes. An aqueous saturated ammonium chloride solution and water were added to the reaction liquid, the mixture was extracted with ethyl acetate, and the organic layer was washed with water, an aqueous saturated ammonium chloride solution and a saturated saline solution. The washed organic layer was dried with anhydrous magnesium sulfate, and filtered and vacuum-concentrated, followed by purifying the residue by silica gel column chromatography (hexane-ethyl acetate) to obtain the title compound as a yellow solid.

### Step 2

### Synthesis of (3R)-3-amino-1-[3,5-bis(trifluoromethyl)benzyl]-3,4-dihydro-1H-1-benzazepin-2,5-dione

Trifluoroacetic acid was added to a solution of tert-butyl{(3R)-1-[3,5-bis(trifluoromethyl)benzyl]-2,5-dioxo-2,3,4,5-tetrahydro-1H-1-benzazepin-3-yl}carbamate, obtained in Step 1, in chloroform, and the mixture was stirred at room temperature for 1 hour. The solvent was distilled off, followed by diluting the residue with chloroform, adding saturated sodium bicarbonate water, and extracting the solution with chloroform. The organic layer was washed with a saturated saline solution, followed by drying the washed organic layer with anhydrous sodium sulfate. After filtration, the solvent was distilled off to obtain the title compound as a yellow solid. This solid was used in the subsequent step without purifying it

### Step 3

### Synthesis of tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate

To a solution of (3R)-3-amino-1-[3,5-bis(trifluoromethyl)benzyl]-3,4-dihydro-1H-1-benzazepin-2,5-dione (2.6 g), obtained in Step 2, in N,N-dimethylformamide, 1-[(tert-butoxycarbonyl]amino]cyclopropanecarboxylic acid, 1-hydroxybenzotriazole monohydrate and 1-(3-dimethylaminopropyl)-3-ethylcarbodiimide hydrochloride were added, and the mixture was stirred overnight at room temperature. An aqueous saturated ammonium chloride solution and water were added to the reaction liquid, the mixture was extracted with ethyl acetate, and the organic layer was washed with water, an aqueous saturated ammonium chloride solution, saturated sodium bicarbonate water and a saturated saline solution. The washed organic layer was dried with anhydrous magnesium sulfate, and filtered and vacuum-concentrated, followed by purifying the residue by silica gel column chromatography (hexane-ethyl acetate) to obtain the title compound as a white solid.

The analytical data of the title compound are shown below.

¹H-NMR(CDCl₃)δ:1.01-1.02(2H,m),1.21-1.23(1H,m),1.50-1.60(10H,m),2.77-2.82(1H,m),3.29-3.34(1H,m),4.95(1H,d,J=15.2 Hz),5.05(1H,d,J=15.2 Hz),5.13(1H,brs),7.17(1H,d,J=7.4 Hz),7.38(1H,t,J=7.4 Hz),7.5.3-7.62(4H,m),7.78(1H,s).

ESI-MS(m/e),600[M+H]⁺

### Example 2

### Synthesis of tert-butyl{1-[({(3S)-1-[3,5-bis(trifluoromethyl)benzyl]-2,5-dioxo-2,3,4,5-tetrahydro-1H-1-benzazepin-3-yl}amino)carbonyl}cyclopropyl}carbamate

Using tert-butyl[(3S)-2,5-dioxo-2,3,4,5-tetrahydro-1H-1-benzazepin-3-yl]carbamate obtained in Reference Example 2, the title compound was obtained as a white solid by the same method as in Example 1.

The analytical data of the title compound are same as in Example 1.

### Example 3

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluorobenzamide

### Step 1

### Synthesis of 1-amino-N-{(3R)-1-[3,5-bis(trifluoromethyl)benzyl]-2,5-dioxo-2,3,4,5,-tetrahydro-1H-1-benzazepin-3-yl}cyclopropane carboxyamide

Trifluoroacetic acid was added to a solution of tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate obtained in Example 1 in chloroform, and the mixture was stirred at room temperature for 2 hours. The solvent was distilled off, followed by diluting the residue with a mixed solution of chloroform/methanol (9:1), adding saturated sodium bicarbonate water, and extracting the solution with chloroform/methanol (9:1). The organic layer was washed with a saturated saline solution, followed by drying the washed organic layer with anhydrous sodium sulfate. After filtration, the solvent was distilled off to obtain the title compound as a white solid. This solid was used in the subsequent step without purifying it.

### Step 2

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluorobenzamide

To a solution of 1-amino-N-{(3R)-1-[3,5-bis(trifluoromethyl)benzyl]-2,5-dioxo-2,3,4,5,-tetrahydro-1H-1-benzazepin-3-yl}cyclopropane carboxyamide (2.5 g), obtained in Step 1, in N,N-dimethylformamide, 4-fluorobenzoate, 1-hydroxybenzotriazole monohydrate and 1-(3-dimethylaminopropyl)-3-ethylcarbodiimide hydrochloride were added, and the mixture was stirred overnight at room temperature. An aqueous saturated ammonium chloride solution and water were added to the reaction liquid, the mixture was extracted with ethyl acetate, and the organic layer was washed with water, an aqueous saturated ammonium chloride solution, saturated sodium bicarbonate water and a saturated saline solution. The washed organic layer was dried with anhydrous magnesium sulfate, and filtered and vacuum-concentrated, followed by purifying the residue by silica gel column chromatography (hexane-ethyl acetate) to obtain the title compound as a white solid.

The analytical data of the title compound are shown below.

¹H-NMR(DMSO-D₆)δ:0.90-0.92(1H,m),1.02-1.04(1H,m),1.19-1.24(1H,m),1.29-1.31(1H,m),3.00-3.02(2H,m),4.96-4.99(1H,m),5.03(1H,d,J=16.0 Hz),5.35(1H,d,J=16.0 Hz),7.24-7.28(2H,m),7.31-7.33(1H,m),7.36-7.38(2H,m),7.57-7.59(1H,m),7.70-7.71(2H,m),7.89-7.93(3H,m),8.06-8.08(1H,m),8.95(1H,brs).

ESI-MS(m/e),622[M+H]⁺

### Example 4

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}benzamide

Using benzoic acid, the title compound was obtained as a white solid by the same method as in Example 3 (Step 2).

The analytical data of the title compound are shown below.

¹H-NMR(CDCl₃)δ:1.20-1.30(2H,m),1.60-1.75(2H,m),2.77-2.93(1H,m),3.39-3.45(1H,m),4.95(1H,d,J=15.2 Hz),5.24-5.29(1H,m),5.65(1H,d,J=15.2 Hz),6.80(1H,brs),7.20-7.23(1H,m),7.30-7.65(9H,m),7.77-7.90(3H,m).

ESI-MS(m/e),604[M+H]⁺

### Example 5

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}pyridin-2-carboxyamide

Using 2-pyridinecarboxylic acid, the title compound was obtained as a white solid by the same method as in Example 3 (Step 2).

The analytical data of the title compound are shown below.

¹H-NMR(DMSO-D₆)δ:0.95-0.98(1H,m),1.10-1.11(1H,m),1.24-1.25(1H,m),1.29-1.31(1H,m),2.99-3.01(2H,m),4.97-4.99(1H,m),5.02(1H,d,J=16.0 Hz),5.35(1H,d,J=16.0 Hz),7.31-7.33(1H,m),7.36-7.39(2H,m),7.57-7.60(2H,m),7.71(2H,s),7.88-7.90(1H,m),7.97-7.99(2H,m),8.06-8.07(1H,m),8.61-8.62(1H,m),9.18(1H,brs).

ESI-MS(m/e),605[M+H]⁺

### Example 6

### Synthesis of N-{(3R)-1-[3,5-bis(trifluoromethyl)benzyl]-2,5-dioxo-2,3,4,5-tetrahydro-1H-1-benzazepin-3-yl}-1-[(3,3,3-trifluoropropanoyl)amino]cyclopropane carboxyamide

Using 3,3,3-trifluoropropionate, the title compound was obtained as a white solid by the same method as in Example 3 (Step 2).

The analytical data of the title compound are shown below.

¹H-NMR(DMSO-D₆)δ:0.76-0.78(1H,m),0.89-0.91(1H,m),1.11-1.14(1H,m),1.27-1.28(1H,m),2.99-3.01(2H,m),3.19-3.22(2H,m),4.97-4.98(1H,m),5.06(1H,d,J=16.0 Hz),5.34(1H,d,J=16.0 Hz),7.32-7.34(1H,m),7.36-7.39(2H,m),7.57-7.60(1H,m),7.72-7.73(2H,m),7.90-7.93(1H,m),7.99-8.01(1H,m),8.78(1H,brs).

ESI-MS(m/e),610[M+H]⁺

### Example 7

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoropyridin-2-carboxyamide

Using 6-fluoropyridin-2-carboxylic acid, the title compound was obtained as a white solid by the same method as in Example 3 (Step 2).

The analytical data of the title compound are shown below

¹H-NMR(DMSO-D₆)δ:0.95-0.97(1H,m),1.11-1.13(1H,m),1.21-1.23(1H,m),1.29-1.30(1H,m),2.99-3.00(2H,m),4.98-4.99(1H,m),5.03(1H,d,J=16.0 Hz),5.34(1H,d,J=16.0 Hz),7.31-7.33(1H,m),7.35-7.41(3H,m),7.57-7.60(1H,m),7.70(2H,s),7.90-7.92(2H,m),8.09-8.17(2H,m),9.08(1H,brs).

ESI-MS(m/e),623[M+H]⁺

### Example 8

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5,6-difluoropyridin-2-carboxyamide

Using 5,6-difluoropyridin-2-carboxylic acid, the title compound was obtained as a white solid by the same method as in Example 3 (Step 2).

The analytical data of the title compound are shown below.

¹H-NMR(DMSO-D₆)δ:0.92-0.95(1H,m),1.10-1.13(1H,m),1.20-1.22(1H,m),1.29-1.31(1H,m),2.98-3.00(2H,m),4.97-5.00(1H,m),5.03(1H,d,J=16.0 Hz),5.34(1H,d,J=16.0 Hz),7.31-7.33(1H,m),7.37-7.38(2H,m),7.58-7.59(1H,m),7.70-7.71(2H,m),7.89-7.91(1H,m),7.96-7.98(1H,m),8.06-8.08(1H,m),8.14-8.16(1H,m),9.11(1H,brs).

ESI-MS(m/e),641[M+H]⁺

### Example 9

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-chloro-3-fluoropyridin-2-carboxyamide

Using 6-chloro-3-fluoropyridin-2-carboxylic acid, the title compound was obtained as a white solid by the same method as in Example 3 (Step 2).

The analytical data of the title compound are shown below.

¹H-NMR(DMSO-D₆)δ:0.94-0.96(1H,m),1.10-1.12(1H,m),1.21-1.23(1H,m),1.32-1.33(1H,m),3.00-3.01(2H,m),4.98-5.01(1H,m),5.05(1H,d,J=16.0 Hz),5.36(1H,d,J=16.0 Hz),7.31-7.33(1H,m),7.37-7.39(2H,m),7.57-7.60(1H,m),7.71-7.72(2H,m),7.75-7.77(1H,m),7.90-7.92(1H,m),7.94-7.97(1H,m),8.05-8.07(1H,m),9.08(1H,brs).

ESI-MS(m/e),657[M+H]⁺

### Example 10

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-2,6-fluoronicotinamide

Using 2,6-difluoronicotinic acid, the title compound was obtained as a white solid by the same method as in Example 3 (Step 2).

The analytical data of the title compound are shown below.

¹H-NMR(DMSO-D₆)δ:0.93-0.95(1H,m),1.05-1.08(1H,m),1.20-1.22(1H,m),1.33-1.35(1H,m),3.02-3.04(2H,m),4.99-5.00(1H,m),5.06(1H,d,J=16.0 Hz),5.35(1H,d,J=16.0 Hz),7.26-7.30(1H,m),7.32-7.34(1H,m),7.37-7.39(2H,m),7.57-7.60(1H,m),7.72-7.73(2H,m),7.89-7.92(1H,m),8.08-8.09(1H,m),8.41-8.43(1H,m),8.98(1H,brs).

ESI-MS(m/e),641[M+H]⁺

### Example 11

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoronicotinamide

Using 6-fluoronicotinic acid, the title compound was obtained as a white solid by the same method as in Example 3 (Step 2).

The analytical data of the title compound are shown below.

¹H-NMR(DMSO-D₆)δ:0.92-0.95(1H,m),1.05-1.08(1H,m),1.21-1.23(1H,m),1.32-1.34(1H,m),3.01-3.03(2H,m),4.99-5.01(1H,m),5.05(1H,d16.0 Hz),5.33(1H,d,J=16.0 Hz),7.27-7.28(1H,m),7.32-7.34(1H,m),7.36-7.38(2H,m),7.58-7.60(1H,m),7.70-7.71(2H,m),7.89-7.91(1H,m),8.19-8.21(1H,m),8.38-8.40(1H,m),8.70-8.73(1H,m),9.13(1H,brs).

ESI-MS(m/e),623[M+H]⁺

### Example 12

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-2-fluoroisonicotinamide

Using 2-fluoroisonicotinic acid, the title compound was obtained as a white solid by the same method as in Example 3 (Step 2).

The analytical data of the title compound are shown below.

¹H-NMR(DMSO-D₆)δ:0.93-0.96(1H,m),1.05-1.07(1H,m),1.21-1.24(1H,m),1.33-1.34(1H,m),3.00-3.02(2H,m),5.00-5.01(1H,m),5.05(1H,d,J=16.0 Hz),5.33(1H,d,J=16.0 Hz),7.32-7.34(1H,m),7.36-7.38(2H,m),7.56-7.61(2H,m),7.70-7.73(2H,m),7.73-7.74(1H,m),7.89-7.92(1H,m),8.22-8.24(1H,m),8.35-8.36(1H,m),9.27(1H,brs).

ESI-MS(m/e),623[M+H]⁺

### Example 13

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluoro-2-(trifluoromethyl)benzamide

Using 4-fluoro-2-(trifluoromethyl)benzenecarboxylic acid, the title compound was obtained as a white solid by the same method as in Example 3 (Step 2).

The analytical data of the title compound are shown below.

¹H-NMR(DMSO-D₆)δ:0.88-0.91(1H,m),0.99-1.02(1H,m),1.17-1.19(1H,m),1.36-1.38(1H,m),3.05-3.07(2H,m),4.99-5.11(2H,m),5.36-5.38(1H,m),7.32-7.41(3H,m),7.60-7.67(3H,m),7.75-7.77(2H,m),7.87-7.89,(3H,m),9.09-9.11(1H,m).

ESI-MS(m/e),690[M+H]⁺

### Example 14

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-methoxybenzamide

Using 4-methoxybenzenecarboxylic acid, the title compound was obtained as a white solid by the same method as in Example 3 (Step 2).

The analytical data of the title compound are shown below.

¹H-NMR(DMSO-D₆)δ:0.89-0.91(1H,m),0.99-1.02(1H,m),1.20-1.22(1H,m),1.28-1.31(1H,m),2,99-3.01(2H,m),3.76(3H,s),5.01-5.05(2H,m),5.34-5.38(1H,m),6.94-6.96(3H,m),7.21-7.38(3H,m),7.55-7.96(6H,m),8.80(1H,brs).

ESI-MS(m/e),634[M+H]⁺

### Example 15

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-chlorobenzamide

Using 4-chlorobenzenecarboxylic acid, the title compound was obtained as a white solid by the same method as in Example 3 (Step 2).

The analytical data of the title compound are shown below.

¹H-NMR(DMSO-D₆)δ:0.90-0.93(1H,m),1.02-1.03(1H,m),1.19-1.24(1H,m),1.29-1.31(1H,m),3.00-3.02(2H,m),4.96-4.99(1H,m),5.03(1H,d,J=16.0 Hz),5.34(1H,d,J=16.0 Hz),7.30-7.32(1H,m),7.35-7.39(2H,m),7.49-7.50(2H,m),7.56-7.60(1H,m),7.70-7.71(2H,m),7.86-7.86(1H,m),7.88-7.90(2H,m),8.08-8.10(1H,m),9.00(1H,brs).

ESI-MS(m/e),638[M+H]⁺

### Example 16

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-1-(trifluoromethyl)cyclopropane carboxyamide

Using 1-(trifluoromethyl)cyclopropanecarboxylic acid, the title compound was obtained as a white solid by the same method as in Example 3 (Step 2).

The analytical data of the title compound are shown below.

¹H-NMR(DMSO-D₆)δ:0.82(1H,s),0.90(1H,s),1.16-1.24(4H,m),1.36-1.38(2H,m),2.97-3.00(2H,m),4.90-4.92(1H,m),5.04-5.08(1H,m),5.35-5.39(1H,m),7.33.7.37(3H,m),7.57-7.59(1H,m),7.64-7.66(1H,m),7.74-7.76(2H,m),7.89-7.91(1H,m),8.31-8.33(1H,m).

ESI-MS(m/e),636[M+H]⁺

### Example 17

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5-fluoropyridin-2-carboxyamide

Using 5-fluoropyridin-2-carboxylic acid, the title compound was obtained as a white solid by the same method as in Example 3 (Step 2).

The analytical data of the title compound are shown below.

¹H-NMR(DMSO-D₆)δ:0.91-0.98(1H,m),1.08-1.13(1H,m),1.19-1.25(1H,m),1.26-1.33(1H,m),2.97-3.08(2H,m),4.93-4.99(1H,m),5.01(1H,d,J=16.0 Hz),5.33(1H,d,J=16.0 Hz),7.30(1H,t,J=7.6 Hz),7.34-7.38(2H,m),7.57(1H,t,J=7.6 Hz),7.69(2H,s),7.83-7.86(1H,m),7.88(1H,s),8.03-8.06(2H,m),8.61(1H,d,J=2.7 Hz),9.16(1H,s).

ESI-MS(m/e),623[M+H]⁺

### Example 18

### Synthesis of 1-ethylpropyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate

Iodomethane was added to a solution of 1-ethylpropyl-1H-imidazol-1-carboxylate in acetonitrile, and the mixture was stirred at room temperature for 24 hours. The solvent was distilled off, followed by adding 1-amino-N-{(3R)-1-[3,5-bis(trifluoromethyl)benzyl]-2,5-dioxo-2,3,4,5-tetrahydro-1H-1-benzazepin-3-yl)cyclopropane carboxamide, obtained in Example 3 (Strep 1), to the residual solution in acetonitrile, and the mixture was stirred overnight at room temperature. The solvent was distilled off, water was added to the residue, and the mixture was extracted with ethyl acetate. The organic layer was washed with a saturated saline solution, followed by drying the washed organic layer with anhydrous magnesium sulfate. Following filtration, the solvent was distilled off, and the residue was purified by silica gel column chromatography (hexane-ethyl acetate) to obtain the title compound as a white solid.

The analytical data of the title compound are shown below.

¹H-NMR(CDCl₃)δ:0.84-1.57(14H,m),2.76(1H,dd,J=19.4,12.7 Hz),3.32(1H,d,J=18.0 Hz),4.62-4.69(1H,m),4.95(1H,d,J=14.9 Hz),5.20-5.23(3H,m),7.11(1H,d,J=8.2 Hz),7.33(1H,t,J=7.4 Hz),7.50-7.53(6H,m),7.74(1H,s).

ESI-MS(m/e),614[M+H]⁺

### Example 19

### Synthesis of N-{(3R)-1-[3,5-bis(trifluoromethyl)benzyl]-2,5-dioxo2,3,4,5-tetrahydro-1H-1-benzazepin-3-yl}-1-{[(tert-butylamino)carbonyl]amino}cyclopropane carboxyamide

To a solution of 1-amino-N-{(3R)-1-[3,5-bis(trifluoromethyl)benzyl]-2,5-dioxo-2,3,4,5,-tetrahydro-1H-1-benzazepin-3-yl}cyclopropane carboxyamide, obtained in Example 3 (Step 1), in chloroform, tert-butylisocyanate was added, and the mixture was stirred overnight at 55° C. The reaction liquid was purified by thin layer column chromatography (hexane-ethyl acetate) to obtain the title compound as a white powder.

The analytical data of the title compound are shown below.

¹H-NMR(CDCl₃)δ:1.04-1.07(2H,brm),1.30(9H,s),1.51-1.54(2H,brm),2.88(1H,s),3.21(1H,d,J=18.0 Hz),5.03(1H,d,J=15.2 Hz),5.13(1H,d,J=15.6 Hz),5.21-5.24(1H,brm),7.10(1H,d,J=7.8 Hz),7.32(1H,t,J=7.4 Hz),7.49-7.75(7H,m).

ESI-MS(m/e),599[M+H]⁺

### Example 20

### Synthesis of tert-butyl[1-({[(3R)-1-(biphenyl-4-ylmethyl)-2,5-dioxo-2,3,4,5-tetrahydro-1H-1-benzazepin-3-yl]amino}carbonyl)cyclopropyl]carbamate

Using 4-(bromomethyl)biphenyl, the title compound was obtained as a white solid by the same method as in Example 1.

The analytical data are shown below.

¹H-NMR(DMSO-D₆)δ:0.84-0.86(2H,m),1.15-1.17(2H,m),1.36(9H,s),3.01-3.03(2H,m),4.88-4.92(2H,m),5.33(1H,d,J=15.6 Hz),7.14-7.16(2H,m),7.30-7.31(2H,m),7.37-7.39(3H,m),7.49-7.51(3H,m),7.56-7.60(4H,m),7.77(1H,brs).

ESI-MS(m/e),540[M+H]⁺

### Example 21

### Synthesis of tert-butyl[1-({[(3S))-1-(biphenyl-4-ylmethyl)-2,5-dioxo-2,3,4,5-tetrahydro-1H-1-benzazepin-3-yl]amino}carbonyl)cyclopropyl]carbamate

Using 4-(bromomethyl)biphenyl, the title compound was obtained as a white solid by the same method as in Example 2.

The analytical data of the title compound are same as in Example 20.

### Example 22

### Synthesis of tert-butyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate

Using 1-(bromomethyl)-4-(trifluoromethoxy)benzene, the title compound was obtained as a white solid by the same method as in Example 1.

The analytical data of the title compound are shown below.

¹H-NMR(DMSO-D₆)δ:0.84-0.86(2H,m),1.15-1.17(2H,m),1.36(9H,s),3.01-3.03(2H,m),4.88-4.92(2H,m),5.33(1H,d,J=15.6 Hz),7.17-7.20(4H,m),7.29-7.33(1H,m),7.38-7.44(2H,m),7.56-7.60(2H,m),7.74(1H,brs).

ESI-MS(m/e),548[M+H]⁺

### Example 23

### Synthesis of tert-butyl{1-[({(3S))-2,5-dioxo-1-[4-(trifluoromethoxy)benzyl]-2,3,4,5-tetrahydro-1H-1-benzazepin-3-yl}amino)carbonyl]cyclopropyl}carbamate

Using 1-(bromomethyl)-4-(trifluoromethoxy)benzene, the title compound was obtained as a white solid by the same method as in Example 2.

The analytical data of the title compound are same as in Example 22.

### Example 24

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide

Using 2-fluorobenzenecarboxylic acid and tert-butyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate, obtained in Example 22, the title compound was obtained as a white solid by the same method as in Example 3.

The analytical data of the title compound are shown below.

¹H-NMR(DMSO-D₆)δ:0.94-0.97(1H,m),1.02-1.03(1H,m),1.25-1.26(1H,m),1.30-1.32(1H,m),3.00-3.01(2H,m),4.86(1H,d,J=15.6 Hz),4.93-4.95(1H,m),5.30(1H,d,J=15.6 Hz),7.17-7.17(4H,m),7.25-7.30(3H,m),7.38(1H,dd,J=8.0,1.6 Hz),7.45(1H,d,J=8.0 Hz),7.48-7.53(1H,m),7.57-7.59(1H,m),7.63-7.65(1H,m),7.83-7.85(1H,m),8.88(1H,brs).

ESI-MS(m/e),570[M+H]⁺

### Example 25

### Synthesis of N-{1-[({(3S)-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide

Using 2-fluorobenzenecarboxylic acid and tert-butyl{1-[({(3S)-2,5-dioxo-1-[4-(trifluoromethoxy)benzyl)-2,3,4,5-tetrahydro-1H-1-benzazepin-3-yl}amino)carbonyl]cyclopropyl}carbamate, obtained in Example 23, the title compound was obtained as a white solid by the same method as in Example 3.

The analytical data of the title compound are same as in Example 24.

### Example 26

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2,4,5-trifluorobenzamide

Using 2,4,5-trifluorobenzenecarboxylic acid, the title compound was obtained as a yellow solid by the same method as in Example 24.

The analytical data of the title compound are shown below.

¹H-NMR(DMSO-D₆)δ:0.93-0.96(1H,m),1.01-1.06(1H,m),1.21-1.25(1H,m),1.30-1.34(1H,m),3.01-3.02(2H,m),4.85(1H,d,J=16.0 Hz),4.93-4.96(1H,m),5.28(1H,d,J=16.0 Hz),7.15-7.20(4H,m),7.30(1H,dd,J=7.4,7.4 Hz),7.38(1H,dd,J=7.4,2.0 Hz),7.44(1H,d,J=7.4 Hz),7.56-7.69(2H,m),7.76-7.79(1H,m),7.91(1H,d,J=7.4 Hz),8.93-8.95(1H,m).

ESI-MS(m/e),606[M±H]⁺

### Example 27

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-4-methoxybenzamide

Using 4-methoxybenzenecarboxylic acid, the title compound was obtained as a yellow solid by the same method as in Example 24.

The analytical data of the title compound are shown below.

¹H-NMR(DMSO-D₆)67 :0.91-0.95(1H,m),0.98-1.02(1H,m),1.24-1.27(2H,m),2.96-2.99,(2H,m),3.76(3H,s),4.82(1H,d,J=15.6 Hz),4.91-4.94(1H,m),5.27(1H,d,J=15.6 Hz),6.94-6.96(2H,m),7.13-7.17(4H,m),7.29-7.31(1H,m),7.36-7.38(1H,m),7.43-7.45(1H,m),7.56-7.59(1H,m),7.83-7.86(3H,m),8.84(1H,s).

ESI-MS(m/e),582[M+H]⁺

### Example 28

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}pyridin-2-carboxyamide

Using pyridin-2-carboxylic acid, the title compound was obtained as a white solid by the same method as in Example 24.

The analytical data of the title compound are shown below.

¹H-NMR(DMSO-D₆)δ:0.97-1.01(1H,m),1.07-1.11(1H,m),1.25-1.28(2H,m),2.97-2.99,(2H,m),4.81(1H,d,J=15.6 Hz),4.92-4.95(1H,m),5.25(1H,d,J=15.6 Hz),7.12-7.16(4H,m),7.30(1H,dd,J=7.4,7.4 Hz),7.37(1H,dd,J=7.8,1.6 Hz),7.44(1H,d,J=7.8 Hz),7.57-7.59(2H,m),7.94-7.98(3H,m),8.61-8.63(1H,m),9.25(1H,brs).

ESI-MS(m/e),553[M+H]⁺

### Example 29

### Synthesis of N-{1-]({(3R)-2,5-dioxo-1-[4-(trifluoromethoxy)benzyl]-2,3,4,5-tetrahydro-1H-1-benzazepin-3-yl}amino)carbonyl]cyclopropyl}-4-fluorobenzamide

Using 4-fluorobenzenecarboxylic acid, the title compound was obtained as a white solid by the same method as in Example 24.

The analytical data of the title compound are shown below.

¹H-NMR(DMSO-D₆)δ:0.92-0.96(1H,m),1.01-1.03(1H,m),1.22-1.31(2H,m),2.92-3.07(2H,m),4.82(1H,d,J=15.6 Hz),4.91-4.97(1H,m),5.27(1H,d,J=15.6 Hz),7.13-7.17(4H,m),7.26-7.30(3H,m),7.36-7.38(1H,m),7.43-7.45(1H,m),7.55-7.60(1H,m),7.92-7.94(3H,m),8.99(1H,brs).

ESI-MS(m/e),570[M+H]⁺

### Example 30

### Synthesis of 4-chloro-N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}benzamide

Using 4-chlorobenzenecarboxylic acid, the title compound was obtained as a white solid by the same method as in Example 24.

The analytical data of the title compound are shown below.

¹H-NMR(DMSO-D₆)δ:0.92-0.96(1H,m),1.00-1.06(1H, m),1.22-1.31(2H,m),2.95-3.03(2H,m),4.82(1H,d,J=16.0 Hz),4.91-4.97(1H,m),5.27(1H,d,J=16.0 Hz),7.13-7.17(4H,m),7.30(1H,dd,J=7.8,7.8 Hz),7.36(1H,dd,J=7.8,1.8 Hz),7.44(1H,d,J=7.8 Hz),7.49-7.52(2H,m),7.55-7.60(1H,m),7.86-7.88(2H,m),7.96(1H,d,J=7.8 Hz),9.04(1H,brs).

ESI-MS(m/e),586[M+H]⁺

### Example 31

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-1-trifluoromethyl)cyclopropane carboxyamide

Using 1-(trifluoromethyl)cyclopropanecarboxylic acid, the title compound was obtained as a yellow solid by the same method as in Example 24.

The analytical data of the title compound are shown below.

¹H-NMR(DMSO-D₆)δ:0.85-0.89(2H,m),1.17-1.23(4H,m),1.35-1.37(2H,m),2.96-2.98(2H,m),4.86-4.88(2H,m),5.30(1H,d,J=15.6 Hz),7.16-7.19(4H,m),7.30(1H,dd,J=7.8,7.8 Hz),7.38(1H,dd,J=7.8,1.6 Hz),7.44(1H,d,J=7.8 Hz),7.55-7.60(2H,m),8.35(1H,brs).

ESI-MS(m/e),584[M+H]⁺

### Example 32

### Synthesis of N-{(3R)-2,5-dioxo-1-[4-(trifluoromethoxy)benzyl]-2,3,4,5-tetrahydro-1H-1-benzazepin-3-yl}-1-[(3,3,3-trifluoropropanoyl)amino]cyclopropane carboxyamide

Using 3,3,3-trifluoropropionic acid, the title compound was obtained as a white solid by the same method as in Example 24.

The analytical data of the title compound are shown below.

¹H-NMR(DMSO-D₆)δ:0.77-0.80(1H,m),0.88-0.91(1H,m),1.14-1.17(1H,m),1.25-1.27(1H,m),2.94-2.98(2H,m),3.21-3.27(2H,m),4.85(1H,d,J=15.6 Hz),4.91-4.97(1H,m),5.26(1H,d,J=15.6 Hz),7.15-7.19,(4H,m),7.30(1H,dd,J=7.6,7.6 Hz),7.37(1H,dd,J=7.6,1.8 Hz),7.43(1H,d,J=7.6 Hz),7.55-7.60(1H,m),7.89(1H,d,J=7.6 Hz),8.80(1H,hrs).

ESI-MS(m/e),558[M+H]⁺

### Example 33

### Synthesis of N-{1-[({(3R)-2,5-dioxo-1-[4-(trifluoromethoxy)benzyl]-2,3,4,5-tetrahydro-1H-1- benzazepin-3-yl}amino)carbonyl]cyclopropyl}-1,3-thiazol-2-carboxyamide

Using 1,3-thiazol-2-carboxylic acid, the title compound was obtained as a white solid by the same method as in Example 24.

The analytical data of the title compound are shown below.

¹H-NMR(DMSO-D₆)δ:0.98-1.01(1H,m),1.09-1.13(1H,m),1.24-1.27(2H,m),2.95-3.04(2H,m),4.82(1H,d,J=15.6 Hz),4.92-4.98(1H,m),5.26(1H,d,J=15.6 Hz),7.13-7.17(4H,m),7.30(1H,dd,J=7.6,7.6 Hz),7.36(1H,dd,J=7.6,1.6 Hz),7.44(1H,d,J=7.6 Hz),7.57-7.59(1H,m),8.00-8.03(3H,m),9.31(1H,brs).

ESI-MS(m/e),559[M+H]⁺

### Example 34

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-6-fluoropyridin-2-carboxyamide

Using 6-fluoropyridin-2-carboxylic acid, the title compound was obtained as a white solid by the same method as in Example 24.

The analytical data of the title compound are shown below.

¹H-NMR(DMSO-D₆)δ:0.95-0.99(1H,m),1.09-1.12(1H,m),1.21-1.30(2H,m),2.92-3.05(2H,m),4.82(1H,d,J=15.6 Hz),4.91-4.97(1H,m),5.26(1H,d,J=15.6 Hz),7.12-7.16(4H,m),7.29-7.30(1H,m),7.35-7.45(3H,m),7.56-7.59(1H,m),7.91-7.94(1H,m),7.96-7.98(1H,m),8.13-8.15(1H,m),9.14(1H,brs).

ESI-MS(m/e),571[M+H]⁺

### Example 35

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-5,6-difluoropyridin-2-carboxyamide

Using 5,6-difluoropyridin-2-carboxylic acid, the title compound was obtained as a white solid by the same method as in Example 24.

The analytical data of the title compound are shown below.

¹H-NMR(DMSO-D₆)δ:0.94-0.98(1H,m),1.08-1.11(1H,m),1.21-1.30(2H,m),2.90-3.04(2H,m),4.82(1H,d,J=15.6 Hz),4.91-4.97(1H,m),5.25(1H,d,J=15.6 Hz),7.13-7.17(4H,m),7.30(1H,dd,J=7.4,7.4 Hz),7.36(1H,dd,J=7.4,1.8 Hz),7.43(1H,d,J=8.2 Hz),7.55-7.60(1H,m),7.94-7.97(2H,m),8.12-8.17(1H,m),9.17(1H,brs).

ESI-MS(m/e),589[M+H]⁺

### Example 36

### Synthesis of 6-chloro-N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-3-fluoropyridin-2-carboxyamide

Using 6-chloro-3-fluoropyridin-2-carboxylic acid, the title compound was obtained as a white solid by the same method as in Example 24.

The analytical data of the title compound are shown below.

¹H-NMR(DMSO-D₆)δ:0.94-0.99(1H,m),1.08-1.11(1H,m),1.25-1.29,(2H,m),2.98-3.00(2H,m),4.84(1H,d,J=15.6 Hz),4.93-4.99(1H,m),5.27(1H,d,J=15.6 Hz),7.13-7.19(4H,m),7.30(1H,dd,J=7.4,7.4 Hz),7.37(1H,dd,J=7.4,1.8 Hz),7.43-7.45(1H,m),7.56-7.60(1H,m),7.74-7.78(1H,m),7.92-7.97(2H,m),9.13(1H,brs).

ESI-MS(m/e),605[M+H]⁺

### Example 37

### Synthesis of N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}benzamide

Using benzoic acid, the title compound was obtained as a white solid by the same method as in Example 24.

The analytical data of the title compound are shown below.

¹H-NMR(DMSO-D₆)δ:0.93-0.97(1H,m),1.02-1.05(1H,m),1.23-1.31(2H,m),2.98-3.03(2H,m),4.82(1H,d,J=15.6 Hz),4.91-4.97(1H,m),5.27(1H,d,J=15.6 Hz),7.12-7.18(4H,m),7.30(1H,dd,J=7.4,7.4 Hz),7.37(1H,dd,J=7.4,1.8 Hz),7.41-7.45(3H,m),7.49-7.51(1H,m),7.55-7.60(1H,m),7.85-7.87(2H,m),7.92(1H,d,J=7.4 Hz),8.98(1H,brs).

ESI-MS(m/e),552[M+H]⁺

### Example 38

### Synthesis of 1-ethylpropyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate

Using tert-butyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate obtained in Example 22, the title compound was obtained as a white solid by the same methods as in Example 3 (Step 1) and Example 18.

The analytical data of the title compound are shown below.

¹H-NMR(CDCl₃)δ:0.87-1.59(14H, m),2.75-2.82(1H,m),3.25-3.35 (1H,br m),4.66(1H,s),4.91-5.17 (4H, m),7.08-7.15(5H,m),7.28-7.32(1H,m),7.45-7.51(2H,m),7.57-7.60(1H,brm).

ESI-MS(m/e),562[M+H]⁺

### Example 39

### Synthesis of tert-butyl{1-[({1-[3,5-bis(trifluoromethyl)benzyl]-7-fluoro-2-oxo-2,3,4,5-tetrahydro-1H-1-benzazepin-3-yl}amino)carbonyl]cyclopropyl}carbamate

Using tert-butyl(7-fluoro-2-oxo-2,3,4,5-tetrahydro-1H-1-benzazepin-3-yl)carbamate obtained in Reference Example 3, the racemic title compound was obtained as a white solid by the same method as in Example 1.

The analytical data of the title compound are shown below.

¹H-NMR(CDCl₃)δ:1.00-1.04(2H,m),1.49(9H,s),1.50-1.58(1H,m),1.68-1.72(1H,m),1.85-1.95(1H,m),2.44-2.49,(2H,m),2.52-2.64(1H,m),4.48-4.55(1H,m),4.86(1H,d,3=14.9 Hz),5.12(1H,brs),5.37(1H,d,J=14.9 Hz),6.90-6.94(1H,m),6.98-7.02(1H,m),7.10-7.15(1H,m),7.30(1H,brs),7.68(2H,s),7.78(1H,s).

ESI-MS(m/e),604[M+H]⁺

### Example 40

### Synthesis of N-{1-[({1-[3,5-bis(trifluoromethyl)benzyl]-7-fluoro-2-oxo-2,3,4,5-tetrahydro-1H-1-benzazepin-3-yl}amino)carbonyl]cyclopropyl}benzamide (enantiomers A and B)

Using tert-butyl{1-[({1-[3,5-bis(trifluoromethyl)benzyl]-7-fluoro-2-oxo-2,3,4,5-tetrahydro-1H-1-benzazepin-3-yl}amino)carbonyl]cyclopropyl}carbamate obtained in Example 39, the title compound was obtained as a racemic body by the same method as in Example 4.

Optical resolution of the obtained racemic body was carried out by chiral column chromatography (Daicel CHIRALPAK AD-H (20*250 mm, 5 um), hexane:ethanol, 7 ml/min) to obtain enantiomer A (faster) and enantiomer B (slower) of the title compound as pale yellow solids, respectively.

The analytical data of the title compound are shown below.

¹H-NMR(CDCl₃)δ:1.04-1.20(2H,m),1.52-1.77(2H,m),1.88-1.96(1H,m),2.18-2.49,(2H,m),2.54-2.63(1H,m),4.48-4.55(1H,m),4.77(1H,d,J=14.9 Hz),5.37(1H,d,J=14.9 Hz),6.79(1H,brs),6.90-6.94(1H,m),6.98-7.02(1H,m),7.10-7.15(1H,m),7.36-7.38(1H,m),7.48-7.51(2H,m),7.62(2H,s),7.78(1H,s),7.80-7.82(2H,m).

EST-MS(m/e),608[M+H]⁺

### Example 41

### Synthesis of N-[1-({[1-[3,5-bis(trifluoromethyl)benzyl]-2-oxo-7-(trifluoromethoxy)-2,3,4,5-tetrahydro-1H-1-benzazepin-3-yl]amino}carbonyl)cyclopropyl]-1-(trifluoromethyl)cyclopropane carboxyamide (enantiomers A and B)

Using tert-butyl(2-oxo-7-(trifluoromethoxy)-2,3,4,5-tetrahydro-1H-1-benzazepin-3-yl)carbamate obtained in Reference Example 4, the title compound was obtained as a racemic body by the same methods as in Example 1 to Example 16.

Optical resolution of the obtained racemic body was carried out by chiral column chromatography (Daicel CHIRALPAK IA (20*250 mm, 5 um), hexane:ethanol, 7 ml/min) to obtain enantiomer A (faster)and enantiomer B (slower) of the title compound as white solids, respectively.

The analytical data of the title compound are shown below.

¹H-NMR(CDCl₃)δ:1.03-1.07(2H,m),1.30-1.32(2H,m),1.56-1.57(4H,m),1.87-1.94(1H,m),2.46-2.51(2H,m),2.62-2.69(1H,m),4.41-4.48(1H,m),4.87(1H,d,J=15.1 Hz),5.37(1H,d,J=15.1 Hz),6.65(1H,s),7.06(1H,s),7.13-7.17(3H,m),7,67(2H,s),7.79(1H,s).

ESI-MS(m/e),706[M+H]⁺

### Example 42

### Synthesis of N-[1-({[1-[3,5-bis(trifluoromethyl)benzyl]-2-oxo-7-(trifluoromethoxy)-2,3,4,5-tetrahydro-1H-1-benzazepin-3-yl}amino}carbonyl)cyclopropyl]benzamide (enantiomers A and B)

Using tert-butyl(2-oxo-7-(trifluoromethoxy)-2,3,4,5-tetrahydro-1H-1-benzazepin-3-yl)carbamate obtained in Reference Example 4, the title compound was obtained as a racemic body by the same methods as in Example 1 to Example 4.

Optical resolution of the obtained racemic body was carried out by chiral column chromatography (Daicel CHIRALPAK IA (20*250 mm, 5 um), hexane:isopropyl alcohol, 7 ml/min) to obtain enantiomer A (faster) and enantiomer B (slower) of the title compound as white solids, respectively.

The analytical data of the title compound are shown below.

¹H-NMR(CDCl₃)δ:1.10-1.17(2H,m),1.59-1.69,(2H,m),1.94-1.96(1H,m),2.43-2.50(2H,m),2.60-2.65(1H,m),4.50-4.57(1H,m),4.84(1H,d,J=15.1 Hz),5.34(1H,d,J=15.1 Hz),6.69(1H,s),7.06(1H,s),7.18(2H,s),7.20-7.30(1H,m),7.4,8(2H,t,J=7.6 Hz),7.57(1H,t,J=7.6 Hz),7.64(2H,s),7.77(1H,$),7.81(2H,d,J=7.6 Hz).

ESI-MS(m/e),674[M+H]⁺

### Example 43

### Synthesis of text-butyl{1-[({1-[3,5-bis(trifluoromethyl)benzyl]-2-oxo-2,3,4,5-tetrahydro-1H-1- benzazepin-3-yl}amino)carbonyl]cyclopropyl}carbamate (enantiomers A and B)

Using tert-butyl(2-oxo-2,3,4,5-tetrahydro-1H-1-benzazepin-3-yl)carbamate obtained in Reference Example 5, the racemic title compound was obtained by the same method as in Example 1, and then optical resolution of the obtained racemic title compound was carried out by optically active column chromatography (Daicel CHIRALPAK AD-H (20*250 mm, 5 um), hexane:isopropyl alcohol, 0.1% diethylamine, 10 ml/min) to obtain enantiomer A (faster) and enantiomer B (slower) as white solids, respectively

The analytical data of the title compound are shown below.

¹H-NMR(CDCl₃)δ:1.00(2H,brs),1.45(9H,s),1.70(1H,brs),1.84-1.95(1H,m),2.44-2.70(1H,m),2.46(2H,brs),4.40-4.58(1H,m),4.89(1H,d,J=14.9 Hz),5.08(1H,brs),5.31(1H,d,J=14.9 Hz),7.10-7.20(2H,m),7.22-7.35(2H,m),7.65(2H,s),7.74(1H,s).

ESI-MS(m/e),586[M+H]⁺

### Example 44

### Synthesis of N-{1-[({3,5-bis(trifluoromethyl)benzyl]-2-oxo-2,3,4,5-tetrahydro-1H-1-benzazepin-3-yl}amino)carbonyl]cyclopropyl}benzamide (enantiomer A)

Using tert-butyl{1-[({1-[3,5-bis(trifluoromethyl)benzyl]-2-oxo-2,3,4,5-tetrahydro-1H-1-benzazepin-3-yl}amino)carbonyl]cyclopropyl}carbamate (enantiomer B) obtained in Example 43, the title compound was obtained as a white solid by the same method as in Example 4.

The analytical data of the title compound are shown below.

¹H-NMR(CDCl₃)δ:1.09-1.16(2H,m),1.60-1.68(2H,m),1.91-1.93(1H,m),2.44-2.49(2H,m),2.60-2.62(1H,m),4.52-4.55(1H,m),4.86(1H,d,J=14.9 Hz),5.37(1H,d,J=14.9 Hz),6.68(1H,s),7.15-7.31(5H,m),7.47(2H,t,J=7.4 Hz),7.47(1H,t,J=7.4 Hz),7.65(2H, s),7.75(1H,s),7.81(2H,d,J=7.4 Hz).

ESI-MS(m/e),590[M+H]⁺

### Example 45

### Synthesis of N-{1-[({1-[3,5-bis(trifluoromethyl)benzyl]-2-oxo-2,3,4,5-tetrahydro-1H-1-benzazepin-3-yl}amino)carbonyl]cyclopropyl}-4-chlorobenzamide

Using tert-butyl{1-[({1-[3,5-bis(trifluoromethyl)benzyl]-2-oxo-2,3,4,5-tetrahydro-1H-1-benzazepin-3-yl}amino)carbonyl]cyclopropyl}carbamate (enantiomer B) obtained in Example 43, the title compound was obtained as a white solid by the same method as in Example 15.

The analytical data of the title compound are shown below.

¹H-NMR(CDCl₃)δ:1.10-1.17(2H,m),1.56-1.61(1H,m),1.66-1.69(1H,m),1.90-1.95(1H,m),2.40-2.58(3H,m),4.50-4.57(1H,m),4.81(1H,d,J=14.8 Hz),5.32(1H,d,J=14.8 Hz),6.90(1H,s),7.15-7.23(3H,m),7.32(1H,t,J=8.0 Hz),7.40-7.43(1H,m),7.44(2H,d,J=8.0 Hz),7.63(2H,s),7.63-7.77(3H,m).

ESI-MS(m/e),624[M+H]⁺

### Example 46

### Synthesis of tert-butyl{1-[({2-oxo-1-[4-(trifluoromethoxy)benzyl]-2,3,4,5-tetrahydro-1H-1-benzazepin-3-yl}amino)carbonyl]cyclobutyl}carbamate (enantiomers A and B)

Using tert-butyl(2-oxo-2,3,4,5-tetrahydro-1H-1-benzazepin-3-yl)carbamate, obtained in Reference Example 5, and 1-[(tert-butoxycarbonyl)amino]cyclobutanecarboxylic acid, the racemic title compound was obtained by the same method as in Example 22, and then optical resolution of the obtained racemic title compound was carried out by optically active column chromatography (Daicel CHIRALPAK AD-H (20*250 mm, 5 um), hexane:ethanol, 10 ml/min) to obtain enantiomer A (faster) and enantiomer B (slower) as white solids, respectively.

The analytical data of the title compound are shown below.

¹H-NMR(DMSO-D₆)δ:1.32(9H,s),1.69-1.94(6H,m),2.24-2.33(3H,m),2.45-2.48(1H,m),4.14-4.17(1H,m),4.83(1H,d,J=14.9 Hz),5.20(1H,d,J=14.9 Hz),7.15-7.37(9H,m),7.50(1H,brs).

ESI-MS(m/e),548[M+H]⁺

### Example 47

### Synthesis of tert-butyl{1-[({2-oxo-1-[4-trifluoromethoxy)benzyl]-2,3,4,5-tetrahydro-1H-1- benzazepin-3-yl}amino)carbonyl]cyclopentyl}carbamate (enantiomers A and B)

Using 1-[(tert-butoxycarbonyl)amino]cyclopentanecarboxylic acid, the racemic title compound was obtained by the same method as in Example 46, and then optical resolution of the obtained racemic title compound was carried out by optically active column chromatography (Daicel CHIRALPAK AD-H (20*250 mm, 5 um), hexane:isopropyl alcohol, 10 ml/min) to obtain enantiomer A (faster) and enantiomer B (slower) as white solids, respectively.

The analytical data of the title compound are shown below.

¹H-NMR(DMSO-D₆)δ:1.26(9H,s),1.49-1.52(4H,m),1.73-1.84(4H,m),2.18-2.20(1H,m),2.37-2.39(1H,m),2.45-2.47(2H,m),4.12-4.15(1H,m),4.83(1H,d,J=15.0 Hz),5.20(1H,d,J=15.0 Hz),7.16-7.19,(4H,m),7.29-7.34(6H,m).

ESI-MS(m/e),562[M+H]⁺

### Example 48

### Synthesis of N-{1-[({7-fluoro-2-oxo-1-[4-(trifluoromethoxy)benzyl]-2,3,4,5-tetrahydro-1H-1-benzazepin-3-yl}amino)carbonyl]cyclopropyl}benzamide (enantiomers A and B)

Using tert-butyl(7-fluoro-2-oxo-2,3,4,5-tetrahydro-1H-1-benzazepin-3-yl)carbamate obtained in Reference Example 3, the racemic title compound was obtained by the same methods as in Example 22 and Example 37, and then optical resolution of the obtained racemic title compound was carried out by optically active column chromatography (Daicel CHIRALPAK IA (20*250 mm, 5 um), hexane:isopropyl alcohol, 10 ml/min) to obtain enantiomer A (faster) and enantiomer B (slower) as white solids, respectively.

The analytical data of the title compound are shown below.

¹H-NMR(DMSO-D₆)δ:0.93-0.99,(2H,m),1.13-1.24(2H,m),1.94-1.97(1H,m),2.11-2.28(2H,m),2.44-2.47(1H,m),4.17-4.19(1H,m),4.71-4.73(1H,m),5.25-5.27(1H,m),7.12-7.21(6H,m),7.46-7.51(4H,m),7.68-7.70(1H,m),7.86-7.88(2H,m),8.93-8.98(1H,m).

ESI-MS(m/e),556[M+H]⁺

### Example 49

### Synthesis of N-{1-[({1-[3,5-(bis(trifluoromethyl)benzyl]-6-chloro-2-oxo-2,3,4,5-tetrahydro-1H-1-benzazepin-3-yl}amino)carbonyl]cyclopropyl}benzamide (enantiomers A and B)

Using tert-butyl(6-chloro-2-oxo-2,3,4,5-tetrahydro-1H-1-benzazepin-3-yl)carbamate obtained in Reference Example 6, the racemic title compound was obtained by the same methods as in Example 39 and Example 40, and then optical resolution of the obtained racemic title compound was carried out by optically active column chromatography (Daicel CHIRALPAK OD-H (20*250 mm, 5 um), hexaneisopropyl alcohol, 10 ml/min) to obtain enantiomer A (faster) and enantiomer B (slower) as white solids, respectively.

The analytical data of the title compound are shown below.

¹H-NMR(DMSO-D6)δ:0.81-1.37(4H,m),1.99-2.34(3H,m),2.93-3.06(1H,brm),4.12-4.20(1H,brm),5.08(1H,d,J=15.6 Hz),5.25(1H,d,J=15.6 Hz),7.24-7.54(6H,m),7.82-7.98(6H,m),8.90-8.86(1H,brm).

ESI-MS(m/e),624[M+H]⁺

### Example 50

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl}cyclopropyl}-1,3-thiazole-2-carboxamide

Using 1,3-thiazole-2-carboxylic acid, the title compound was obtained as a white solid by the same method as in Example 3 (Step 2).

The analytical data of the title compound are shown below.

¹H-NMR(Acetone-D₆)δ:1.21-1.23(2H,m),1.47-1.52(2H,m),2.88-2.94(1H,m),3.21-3.26(1H,m), 5.19-5.22(2H,m),5.49-5.52(1H,d, J=16.0 Hz),7.39(1H,t,J=7.4 HZ), 7.51-7.54(2H,m), 7.63(1H,t,J=7.4 HZ),7.85(3H,m),7.89(1H,s), 7.96(1H,s), 7.99(1H,s),8.68(1H,s).

ESI-MS(m/e),611[M+H]⁺

### Example 51

### Synthesis of tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}carbamate

Using 1-[(tert-butoxycarbonyl]amino]cyclobutanecarboxylic acid, the title compound was obtained as a white solid by the same method as in Example 1 (Step 3).

The analytical data of the title compound are shown below.

¹H-NMR(Acetone-D6) δ: 1.42(9H,s),1.87-1.97(2H,m),2.07-2.14(2H,m) 2.52-2.54(1H,m),2.66-2.69(1H,m),2.84-2.87(1H,m),3.26-3.31(1H,m),5.15-5.16(1H,m), 5.24(1H,d,J=15.8 Hz),5.52(1H,d,J=15.8 Hz),6.83(1H,bs),7.41(1H,t,J=7.8 Hz),7.52(1H,d,J=8.0 Hz), 7.56(1H,d,J=7.8 Hz), 7.65(1H,t,J=8.0 Hz) 7.89(2H,s), 7.93(1H,s).

ESI-MS(m/e),614[M+H]⁺

### Example 52

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}-1,3-thiazole-2-carboxamide

To a solution of 1-amino-N-{(3R)-1-[3,5-bis(trifluoromethyl)benzyl]-2,5-dioxo-2,3,4,5,-tetrahydro-1H-1-benzazepin-3-yl}cyclobutylamine trifluoroacetic acid salt, obtained in Reference Example 7, in dichloromethane,1,3-thiazole-2-carbonyl chloride, 4-dimethylaminopyridine and N,N-diisopropylethyl amine were added, and the mixture was stirred for 30 min. at room temperature. An aqueous saturated ammonium chloride solution was added to the reaction liquid, the mixture was extracted with ethyl acetate, and the organic layer was washed with, an aqueous saturated ammonium chloride solution, saturated sodium bicarbonate water and a saturated saline solution. The washed organic layer was dried with anhydrous magnesium sulfate, and filtered and vacuum-concentrated, followed by purifying the residue by silica gel column chromatography (hexane-ethyl acetate) to obtain the title compound as a white solid. The analytical data of the title compound are shown below.

¹H-NMR(CDCl₃) δ: 2.04-2.09(2H,m),2.35-2.43(2H,m),2.80-2.89(3H,m) 3.37-3.42(1H,m),4.95(1H,d,J=15.6 Hz),5.24-5.29(1H,m),5.31(1H,d,J=15.6 Hz),7.18 (1H,d,J=8.2 Hz),7.36(1H,t,J=7.5 Hz),7.55(3H,m),7.63(1H,d,J=2.8 Hz),7.67 (1H,d,J=6.4 Hz), 7.77(1H,s), 7.80(1H,s), 7.90(1H,d,J=2.8 Hz).

ESI-MS(m/e),625[M+H]⁺

### Example 53

### Synthesis of N-{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}-4-fluorobenzamide

Using 4-fluorobenzoyl chloride, the title compound was obtained as a white solid by the same method as in Example 52.

The analytical data of the title compound are shown below.

¹H-NMR(CDCl₃) δ: 2.03-2.09(2H,m),2.34-2.41(2H,m),2.79-2.89(3H,m) 3.38-3.42(1H,m),4.97(1H,d,J=15.5 Hz),5.23-5.27(1H,m),5.29(1H,d,J=15.5 Hz),6.64(1H,s),7.13-7.19(2H,m),7.37(1H,t,J=7.6 Hz),7.56-7.58(3H,m),7.75(1H,d,J=6.5 Hz),7.78(1H,s),7.82-7.85(2H, m)

ESI-MS(m/e),636[M+H]⁺

Reference examples will now be described below.

### Reference Example 1

### Synthesis of tert-butyl[(3R)-2,5-dioxo-2,3,4,5-tetrahydro-1H-1-benzazepin-3-yl]carbamate

### Step 1

### Synthesis of (2S)-4-(2-aminophenyl)-2-[(tert-butoxycarbonyl)amino]-4-oxoethyl acetate

Sodium hydrogen carbonate and dicarbonic acid di-tert-butyl were added to a mixed suspension of D-kynurenine in tetrahydrofuran and water, and the mixture was stirred overnight at room temperature. A 5N aqueous hydrochloric acid solution and water were added to the reaction liquid, and the mixture was extracted with ethyl acetate. The organic layer was washed with a saturated saline solution, then dried with anhydrous magnesium sulfate, and filtered, followed by distilling the solvent off. Chloroform was added to the residue, and the precipitated solid was thus obtained by filtration and dried to obtain the title compound as a yellow solid.

### Step 2

Synthesis of tert-butyl[(3R)-2,5-dioxo-2,3,4,5-tetrahydro-1H-1-benzazepin-3-yl]carbamate

To a solution of (2S)-4-(2-aminophenyl)-2-[(tert-butoxycarbonyl]amino]-4-oxoethyl acetate (2) (7.1 g) in N,N-dimethylformamide, l-hydroxybenzotriazole monohydrate and 1-(3-dimethylaminopropyl)-3-ethylcarbodiimide hydrochloride were added, and the mixture was stirred overnight at room temperature. A saturated aqueous ammonium chloride solution and water were added to the reaction liquid, and the resultant precipitate was obtained by filtration, washed with hexane/ethyl acetate (=2:1), and dried, followed by obtaining the title compound as a white solid.

### Reference Example 2

### Synthesis of tert-butyl[(3R)-2,5-dioxo-2,3,4,5-tetrahydro-1H-1-benzazepin-3-yl]carbamate

Using L-kynurenine, the title compound was obtained as a white solid by the same method as in Example 1.

### Reference Example 3

### Synthesis of tert-butyl(7-fluoro-2-oxo-2,3,4,5-tetrahydro-1H-1-benzazepin-3-yl)carbamate

### Step 1

### Synthesis of 3-azido-7-fluoro-1,3,4,5-tetrahydro-2H-1-benzazepin-2-on

N,N,N′N′-tetramethylethylenediamine and iodotrimethylsilane were added to a solution of 7-fluoro-1,3,4,5-tetrahydro-2H-1-benzazepin-2-on in dichloromethane at −15° C., and the mixture was stirred at the same temperature for 15 minutes, followed by adding iodine and thus stirring the mixture at −15° C. for 30 minutes. A 5% aqueous sodium thiosulfate solution was added, and the mixture was extracted with ethyl acetate. The organic layer was washed with water and a saturated saline solution, and the washed organic layer was dried with anhydrous magnesium sulfate, filtered and vacuum-concentrated. The residue was dried under reduced pressure to obtain 3-iodo-7-fluoro-1,3,4,5-tetrahydro-2H-1-benzazepin-2-on as a white solid. Sodium azide was added to a solution of this crude product in dimethylsulfoxide, and the mixture was stirred overnight at room temperature. Water was added to the reaction solution, and the resultant precipitate was obtained by filtration and then washed with water, and dried to obtain 3-azido-7-fluoro-1,3,4,5-tetrahydro-2H-1-benzazepin-2-on as a white solid.

This product was used in the subsequent step without purification.

### Step 2

### Synthesis of 3-amino-7-fluoro-1,3,4,5-tetrahydro-2H-1-benzazepin-2-on

To a solution of 3-azido-7-fluoro-1,3,4,5-tetrahydro-2H-1-benzazepin-2-on in methanol and chloroform, 10% palladium-carbon was added under nitrogen atmosphere, and the mixture was stirred under hydrogen atmosphere at room temperature for 5 hours. The reaction liquid was Celite-filtered and vacuum-concentrated. The residue was washed with chloroform to obtain 3-amino-7-fluoro-1,3,4,5-tetrahydro-2H-1-benzazepin-2-on (5.36 g) as a white solid.

### Step 3

### Synthesis of tert-butyl(7-fluoro)-2-oxo-2,3,4,5-tetrahydro-1H-1-benzazepin-3-yl)carbamate

Triethylamine and di-tert-butyl dicarbonate were added to a solution of 3-amino-7-fluoro-1,3,4,5-tetrahydro-2H-1-benzazepin-2-on in ethyl acetate and methanol, and the mixture was stirred at room temperature for 1 hour. The reaction liquid was diluted with ethyl acetate, followed by washing with 10% aqueous citric acid, water and a saturated saline solution, then drying with anhydrous magnesium sulfate, filtration, and then distilling the solvent off to obtain tert-butyl(7-fluoro-2-oxo-2,3,4,5-tetrahydro-1H-1-benzazepin-3-yl)carbamate as a colorless oil.

This product was used in the subsequent step without purification.

### Reference Example 4

### Synthesis of tert-butyl(2-oxo-7-(trifluoromethoxy)-2,3,4,5-tetrahydro-1H-1-benzazepin-3-yl)carbamate

Using 7-(trifluoromethoxy)-1,3,4,5-tetrahydro-2H-1-benzazepin-2-on, the title compound was obtained as a colorless oil by the same method as in Reference Example 3.

### Reference Example 5

### Synthesis of tert-butyl(2-oxo-2,3,4,5-tetrahydro-1H-1-benzazepin-3-yl)carbamate

Using 1,3,4,5-tetrahydro-2H-1-benzazepin-2-on, the title compound was obtained as a colorless oil by the same method as in Reference Example 3.

### Reference Example 6

### Synthesis of tert-butyl(6-chloro-2-oxo-2,3,4,5-tetrahydro-1H-1-benzazepin-3-yl)carbamate

Using 6-chloro-1,3,4,5-tetrahydro-2H-1-benzazepin-2-on, the title compound was obtained as a colorless oil by the same method as in Reference Example 3.

### Reference Example 7

### Synthesis of 1-ammonium-N-{(3R)-1-[3,5-bis(trifluoromethyl)benzyl]-2,5-dioxo-2,3,4,5,-tetrahydro-1H-1-benzazepin-3-yl}cyclobutyl carboxamide trifluoroacetic acid salt

Trifluoroacetic acid was added to a solution of tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}carbamate obtained in Example 19 in dichloromethane, and the mixture was stirred at room temperature for 1 hour. The solvent was distilled off and the residue was reconstituted in dichloromethane. The solvent was again distilled off.

The usefulness of the compound represented by formula (I) as a medicament is proved, for example, in the test described below.

### Cloning of Human DGAT1 Gene and Expression in Yeast

Human DGAT1 genes were amplified by PCR using primers described below from human cDNA library (Clontech).

DGAT1F: 5′-ATGGGCGACCGCGGCAGCTC-3′
DGAT1R: 5′-CAGGCCTCTGCCGCTGGGGCCTC-3′

The amplified human DGAT1 genes were introduced into a yeast expression vector pPICZA (Invitrogen). The resultant expression plasmid was introduced into a yeast (Pichia pastris) by electroporation to produce a recombinant yeast. The recombination yeast was cultured in the presence of 0,5% methanol for 72 hours, and the cells were crushed using glass beads in 10 mM Tris pH 7.5, 250 mM sucrose and 1 mM EDTA, followed by adjusting the membrane fraction by centrifugation to use the adjusted membrane fraction as an enzyme source.

### DGAT1 Inhibitory Activity Test

To the reaction liquid having the following composition: 100 mM Tris pH 7.5, 100 mM MgCl₂, 100 mM sucrose, 40 μM dioelin, 15 μm [¹⁴C]-oleoyl-CoA, 0.25 μg of test substance, DGAT1-expressed yeast membrane fraction, was added, and the mixture having a volume of 100 μl was incubated at room temperature for 30 minutes. To the reaction liquid, 100 μl of 2-propanol/heptan/H₂O (80:20:2) was added, the mixture was stirred well, followed by adding 200 μl of heptane and further stirring the mixture. After centrifugation, the heptane layer was collected, ethanol/0.1N NaOH/H₂O (50:5:45) was added, the mixture was thus stirred, followed by recentrifuging the mixture and collecting the heptane layer. After exsiccation of the obtained heptane layer, 100 μl of Microscint 0 (PerkinElmer) was added, and the radioactivity was measured with a liquid scintillation counter. The inhibitory activity was calculated from the following formula:

Inhibition rate=100−(radioactivity in case of addition of test compound−background)/(radioactivity in case of addition of no test compound−background)×100

wherein the background means the radioactivity in case of addition of no membrane fraction.

The DGAT1 inhibitory activity of the compound according to an embodiment of the present invention by the aforementioned method is shown below.

TABLE 5

| | DGAT1 Inhibitory Activity |
|---|---|
| Example number | IC₅₀ (nM) |
| Example 1 | 37 |
| Example 2 | 373 |
| Example 3 | 140 |
| Example 6 | 442 |
| Example 8 | 85 |
| Example 19 | 221 |
| Example 21 | 24 |
| Example 41 Enantiomer A | 137 |
| Example 41 Enantiomer B | 858 |
| Example 46 Enantiomer A | 189 |
| Example 46 Enantiomer B | 14 |
| Example 47 Enantiomer A | 153 |
| Example 47 Enantiomer B | 21 |

TABLE 6

| | DGAT1 Inhibitory Activity |
|---|---|
| Example number | IC₅₀ (nM) |
| Example 50 | 65 |
| Example 51 | 41 |
| Example 52 | 253 |
| Example 53 | 77 |

As shown in Table 5 and Table 6, the compound according to an embodiment of the present invention has excellent DGAT1 inhibitory activity in consideration of an index of IC₅₀.

1. A compound of formula I 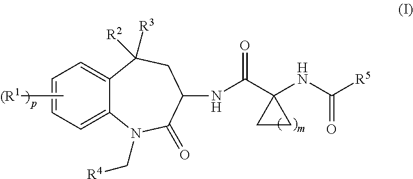
wherein:
R¹ is each independently selected from the group consisting of:
(1) halogen atom,
(2) lower alkyl group unsubstituted or substituted with one to three halogen atoms, and
(3) lower alkoxy group unsubstituted or substituted with one to three halogen atoms,
R² and R³ are each independently hydrogen atoms, or R² and R³ taken together form an oxo group;
R⁴ is selected from: 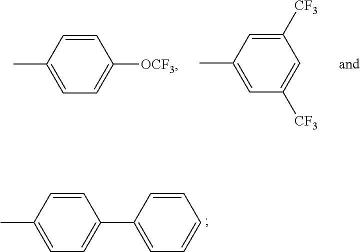
R⁵ is
(1) a group selected from the group consisting of phenyl, pyridinyl and thiazolyl,
or
(2) a group selected from the group consisting of: 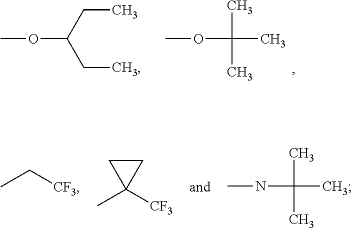
wherein phenyl, pyridinyl and thiazoly are unsubstituted or substituted with one to three halogen atoms, lower alkoxy groups or trifluoromethyl groups;
m is an integer of from 1 to three;
p is an integer of from 0 to four;
or a pharmaceutically acceptable salt thereof.

2. The compound according to claim 1, wherein: formula I is represented by formula I-1: 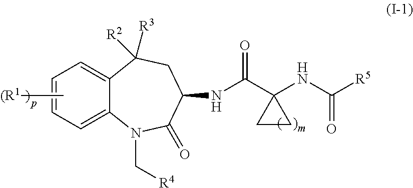
wherein: R¹, R², R³, R⁴, R⁵, p and m have the same meaning as the above;
or a pharmaceutically acceptable salt thereof.

3. The compound according to claim 2, wherein:
R⁴ is: 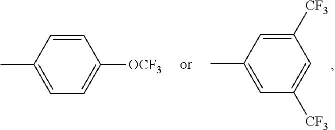
or a pharmaceutically acceptable salt thereof.

4. The compound according to claim 3, wherein: m is 1;
or a pharmaceutically acceptable salt thereof.

5. The compound according to claim 4, wherein: p is 0 or 1, and R¹ is selected from the group consisting of: chlorine, fluorine and trifluoromethoxy group;
or a pharmaceutically acceptable salt thereof.

6. The compound according to claim 5, wherein: R² and R³ taken together form an oxo group; or a pharmaceutically acceptable salt thereof.

7. The compound according to claim 6, wherein: R⁵ is selected from the group consisting of: tert-butoxy, phenyl, 4-chlorophenyl, 4-fluorophenyl, 2-pyridinyl, 6-fluoro-2-pyridinyl, 5,6-difluoro-2-pyridinyl, 6-chloro-3-fluoro-2-pyridinyl and 2,6-difluoro-3-pyridinyl; or a pharmaceutically acceptable salt thereof.

8. The compound according to claim 1, wherein: the compound represented by formula I is:
tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate; tert-butyl{1-[({(3S))-1-[3,5-bis(trifluoromethyl)benzyl]-2,5-dioxo-2,3,4,5-tetrahydro-1H-1-benzazepin-3-yl}amino)carbonyl]cyclopropyl}carbamate; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluorobenzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}benzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}pyridin-2-carboxyamide; N-{(3R)-1-[3,5-bis(trifluoromethyl)benzyl]-2,5-dioxo-2,3,4,5-tetrahydro-1H-1-benzazepin-3-yl}-1-[(3,3,3-trifluoropropanoyl)amino]cyclopropane carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5,6-difluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-chloro-3-fluoropyridin-2-carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-2,6-difluoronicotinamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-6-fluoronicotinamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-2-fluoroisonicotinamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-fluoro-2-(trifluoromethyl)benzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-4-methoxybenzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-1-(trifluoromethyl)cyclopropane carboxyamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-5-fluoropyridin-2-carboxyamide; 1-ethylpropyl{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}carbamate; N-{(3R)-1-[3,5-bis(trifluoromethyl)benzyl]-2,5-dioxo-2,3,4,5-tetrahydro-1H-1-benzazepin-3-yl}-1-{[(tert-butylamino)carbonyl]amino}cyclopropane carboxyamide; tert-butyl[1-([(3R)-1-(biphenyl-4-ylmethyl)-2,5-dioxo-2,3,4,5-tetrahydro-1H-1-benzazepin-3-yl]amino}carbonyl)cyclopropyl]carbamate; tert-butyl[1-({[(3S))-1-(biphenyl-4-ylmethyl)-2,5-dioxo-2,3,4,5-tetrahydro-1H-1-benzazepin-3-yl]amino}carbonyl)cyclopropyl]carbamate; tert-butyl{1-[({(3R)-2,5-dioxo-1-[4-(trifluoromethoxy)benzyl]-2,3,4,5-tetrahydro-1H-1-benzazepin-3-yl}amino)carbonyl]cyclopropyl}carbamate; tert-butyl{1-[({(3S))-2,5-dioxo-1-[4-(trifluoromethoxy)benzyl]-2,3,4,5-tetrahydro-1H-1-benzazepin-3-yl}amino)carbonyl]cyclopropyl}carbamate; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide; N-{1-[({(3S))-2,5-dioxo-1-[4-(trifluoromethoxy)benzyl]-2,3,4,5-tetrahydro-1H-1-benzazepin-3-yl}amino)carbonyl]cyclopropyl}-2-fluorobenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-2,4,5-trifluorobenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-4-methoxybenzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}pyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-4-fluorobenzamide; 4-chloro-N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}benzamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-1-(trifluoromethyl)cyclopropane carboxyamide; N-{(3R)-2,5-dioxo-1-[4-(trifluoromethoxy)benzyl]-2,3,4,5-tetrahydro-1H-1-benzazepin-3-yl}-1-[3,3,3-(trifluoropropanoyl)amino]cyclopropane carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-1,3-thiazol-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-6-fluoropyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-5,6-difluoropyridin-2-carboxyamide; 6-chloro-N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}-3-fluoropyridin-2-carboxyamide; N-{1-[({(3R)-2,5-dioxo-1-[4-(trifluoromethoxy)benzyl]-2,3,4,5-tetrahydro-1H-1-benzazepin-3-yl}amino)carbonyl]cyclopropyl}benzamide; 1-ethylpropyl{1-[({(3R)-2,5-dioxo-1-[4-(trifluoromethoxy)-benzyl]-2,3,4,5-tetrahydro-1H-1-benzazepin-3-yl}amino)carbonyl]cyclopropyl}carbamate; tert-butyl{1-[({1-[3,5-bis(trifluoromethyl)benzyl)-7-fluoro-2-oxo-2,3,4,5-tetrahydro-1H-1-benzazepin-3-yl}amino)carbonyl]cyclopropyl}carbamate; N-{1-[({1-[3,5-bis(trifluoromethyl)benzyl)-7-fluoro-2-oxo-2,3,4,5-tetrahydro-1H-1-benzazepin-3-yl}amino)carbonyl]cyclopropyl}benzamide; N-[1-({[1-[3,5-bis(trifluoromethyl)benzyl)-2-oxo-7-(trifluoromethoxy)-2,3,4,5-tetrahydro-1H-1-benzazepin-3-yl]amino}carbonyl)cyclopropyl]-1-(trifluoromethyl)cyclopropane carboxyamide; N-[1-({[1-[3,5-bis(trifluoromethyl)benzyl)-2-oxo-7-(trifluoromethoxy)-2,3,4,5-tetrahydro-1H-1-benzazepin-3-yl]amino}carbonyl)cyclopropyl]benzamide; tert-butyl{1-[({1-[3,5-bis(trifluoromethyl)benzyl]-2-oxo-2,3,4,5-tetrahydro-1H-1-benzazepin-3-yl}amino)carbonyl]cyclopropyl}carbamate; N-{1-[({1-[3,5-bis(trifluoromethyl)benzyl)-2-oxo-2,3,4,5-tetrahydro-1H-1-benzazepin-3-yl}amino)carbonyl]cyclopropyl}benzamide (enantiomer A); N-{1-[({1-[3,5-bis(trifluoromethyl)benzyl]-2-oxo-2,3,4,5-tetrahydro-1H-1-benzazepin-3-yl}amino)carbonyl]cyclopropyl}-4-chlorobenzamide; tert-butyl{1-[({2-oxo-1-[4-(trifluoromethoxy)benzyl]-2,3,4,5-tetrahydro-1H-1-benzazepin-3-yl}amino)carbonyl]cyclobutyl}carbamate; tert-butyl{1-[({2-oxo-1-[4-(trifluoromethoxy)benzyl]-2,3,4,5-tetrahydro-1H-1-benzazepin-3-yl}amino)carbonyl]cyclopentyl}carbamate; N-{1-[({7-fluoro-2-oxo-1-[4-(trifluoromethoxy)benzyl]-2,3,4,5-tetrahydro-1H-1-benzazepin-3-yl}amino)carbonyl]cyclopropyl}benzamide; and N-{1-[({1-[3,5-bis(trifluoromethyl)benzyl)-6-chloro-2-oxo-2,3,4,5-tetrahydro-1H-1-benzazepin-3-yl}amino)carbonyl]cyclopropyl}benzamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclopropyl}-1,3-thiazole-2-carboxamide; tert-butyl{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}carbamate; N-{1-[({(3R)-1-[3,5-bis(trifluoromethyl)benzyl]-2,5-dioxo-2,3,4,5,-tetrahydro-1H-1-benzazepin-3-yl}cyclobutyl}-1,3-thiazole-2-carboxamide; N-{1-[({(3R)-1-[3,5-bis(trifluoromethyl)benzyl]-2,5-dioxo-2,3,4,5-tetrahydro-1H-1-benzazepin-3-yl}amino)carbonyl]cyclobutyl}-4-fluorobenzamide.

9-10. (canceled)

11. A pharmaceutical composition comprising a compound of claim 1, or a pharmaceutically acceptable salt thereof and a pharmaceutically acceptable carrier.

12. A method of treating diabetes comprising administering to a patient in need thereof a therapeutically effective amount of a compound of claim 1, or a pharmaceutically acceptable salt thereof.
